(19) United States
(12) Patent Application Publication
(10) Pub. No.: US 2021/0347895 A1
(43) Pub. Date: Nov. 11, 2021

(54) EXOSOME-TARGETING BISPECIFIC ANTIBODIES

(71) Applicant: IMMUNOME, INC., Exton, PA (US)

(72) Inventors: Matthew K. ROBINSON, Blue Bell, PA (US); Michael John MORIN, Salisbury, MA (US)

(21) Appl. No.: 17/283,614

(22) PCT Filed: Oct. 17, 2019
(86) PCT No.: PCT/US2019/056698

§ 371 Date: Apr. 8, 2021

Related U.S. Application Data
(60) Provisional application No. 62/746,862, filed on Oct. 17, 2018.

(51) Int. Cl. C07K 16/28

(52) U.S. Cl. CPC C07K 16/2827; A61K 2039/545; C07K 2317/565; C07K 2317/31

(57) ABSTRACT

The inventions described herein are directed to bispecific antibodies that are capable of selectively targeting exosomes by specifically binding a first exosome-associated protein and Programmed Death Ligand-1 (“PD-L1”) as a second exosome-associated protein. These bispecific antibodies can disrupt the suppression of anti-tumor activity by immune cells by targeting tumor-cell derived exosomes that inhibit T cell activation. Therefore, bispecific antibodies of the invention can be used in methods for treating cancers.

### FIELD OF THE INVENTION

The field of this invention relates to antibody-based therapeutics for the treatment of cancer.

### BACKGROUND

The human adaptive immune system responds to antigenic challenge through both cellular (T cell) and humoral (B cell) processes. The humoral response results in selection and clonal amplification of B cells that express surface bound immunoglobulin (Ig) molecules capable of binding to antigens. T cells develop from immature precursors that originate in the bone marrow and then migrate to the thymus, where they proliferate and differentiate into mature T lymphocytes.

The development of a humoral response includes the processes of somatic hypermutation and class switching take place concordant with the clonal amplification. Together, these processes lead to secreted antibodies that have been affinity matured against a target antigen and contain a constant domain belonging to one of the four general classes (M, D, A, G, or E). Each class of antibody (IgM, IgD, IgA, IgG, and IgE) interact in distinct ways with the cellular immune system. Hallmarks of antibodies that have been affinity matured against a target antigen can include: 1) nucleotide, and subsequent amino acid, changes relative to the germline gene, 2) high binding affinity for the target antigen, 3) binding selectivity for the target antigen as compared to other proteins.

It is well understood that oncology patients can mount an immune response against tumor cell antigens. Those antigens can result either from genetic changes within the tumor that lead to mutated proteins or aberrant presentation of otherwise normal proteins to the immune system. Aberrant presentation may occur through processes that include, but are not limited to, ectopic expression of neonatal proteins, mis-localization of intracellular proteins to the cell surface, or lysis of cells. Aberrant expression of enzymes that lead to changes in glycosylation of proteins can also result in generation of non-self antigens that are recognized by the humoral immune system.

Antibodies that bind selectively to disease-related proteins, including those related to cancer, have proven successful at modulating the functions of their target proteins in ways that lead to therapeutic efficacy. The ability of the human immune system to mount antibody responses against mutated, or otherwise aberrant, proteins suggests that patients' immune responses may include antibodies that are capable of recognizing, and modulating the function of, critical tumor-drivers. In that regard, the increased expression of proteins involved in cell membrane trafficking is associated with increased tumor growth and tumor metastasis.

Membrane trafficking contributes to the regulation of a wide range of cellular processes. Internalization of cell surface receptors is a critical mechanism for appropriate modulation of growth factor receptor mediated signaling. Internalization via clathrin-coated vesicles represents one pathway for internalization of cancer relevant receptors from the cell surface. Loading of those receptors into clathrin-coated pits (CCPs), for subsequent internalization in clathrin-coated vesicles, is one of the first steps in the pathway. Loading of receptors into CCPs is dictated in part by interaction with adaptor molecules, such as Epsin-1 (EPN1).

EPN1 is an approximately 60.3 kDa protein that localizes to cellular membranes. It contains a PI(4,5)P2-, ubiquitin-, and clathrin/AP-2-interacting domains. Knocking down expression of endogenous expression of EPN1, overexpressing mutant forms of EPN1, or treating cells with agents designed to block interaction of EPN1 with its cargo molecules can inhibit internalization of known CCP-dependent cargo. Examples of such cargo are VEGFR and ERBB3. Notably, certain types of cancer tumor cells release EPN1-loaded exosomes, and the growth of such cells can be blocked by preventing EPN1 from interacting with its receptor.

Naïve, mature, T cells leave the thymus and migrate to specialized lymphoid organs, such as lymph nodes, spleen, and the tonsils. If a naïve T cell receives an activation signal, it undergoes multiple rounds of divisions to yield populations of effector cells, as well as other cells that revert to a quiescent phase in which they remain primed to respond to a subsequent exposure to the activation signal.

Activation of T cells occurs through a two-signal co-stimulation model (FIG. 21). The primary signal for T cell activation is the binding of a T cell receptor (TCR) on the surface of a T cell to its cognate antigen (Ag) that is presented on the surface of an antigen presenting cell (“APC”), in a complex with a major histocompatibility-complex (“MHC”) protein. This mode of activation, in addition to allowing for response to foreign antigens, also allows for self versus non-self discrimination, and the achievement of immune tolerance.

The second activation signal is transduced to the T lymphocyte through co-stimulatory molecules present on the surface of APCs. Interplay between the strengths of the primary and secondary signals is necessary for appropriate T cell activation. Lack of co-stimulation, in the presence of antigenic activation, can lead to T cell exhaustion or tolerance to foreign antigen stimulation. In contrast, strong primary signaling through the TCR can overcome lack of co-stimulation.

Activation of T cells through co-stimulation is also balanced by negative co-stimulatory signals. The interplay between positive and negative co-stimulatory signals provides for proper balance of immune activation against foreign antigens while preventing the breaking of tolerance and development of autoimmunity.

Molecules responsible for co-stimulation are of therapeutic interest because manipulation of signaling through those molecules can either enhance or dampen T cell responses. T cell exhaustion, or anergy, is correlated with expression of programmed cell death 1 (PD-1) on the surface of T cells. Binding of the ligand programmed cell death-ligand 1 (PD-L1) to its cognate receptor, PD-1, reduces T cell activation. Antagonizing the PD-1/PD-L1 pathway, with antibodies capable of preventing binding of PD-L1 to PD-1, has been demonstrated to enhance activation of T cells and improve clinical outcome of oncology patients.

PD-L1, present on tumor-derived exosomes, represents a potent negative-regulatory signal for T cells. Exosomes are nano-sized (30-150 nm) membrane vesicles derived from multivesciular bodies and secreted into the extracellular environment. Exosomes contain cell-derived, membrane-bound receptors and ligands, as well as intracellular components such as RNA and metabolites. Tumor cells are known to produce exosomes, which are capable of transferring, at a distance, tumor-derived components to normal cells. Tumor-derived exosomes have been linked to, among other things, transformation of normal cells and conditioning of the metastatic niche.

Increased levels of exosome-associated PD-L1 is a marker for advanced disease and may inversely correlate with clinical outcome in certain cancers, including head and neck cancer, gastric cancer, melanoma, and glioblastoma multiforma. Disruption of the exosome-induced T cell supression in tumors represents a therapeutic strategy for the treatment of cancer. With that aim in mind, bispecific antibodies, capable of targeting exosomal PD-L1 and another exosome marker are described herein as effective agents for overcoming PD-L1 induced immune supression and treating various cancers. More particularly, bispecific antibodies which target PD-L1 and EPN1 are disclosed and exemplified herein.

### SUMMARY OF THE INVENTION

The invention described herein is directed to bispecific antibodies that are capable of simultaneously targeting exosomes by specifically binding a first exosome-associated protein and Programmed Death Ligand-1 (“PD-L1”) as a second exosome-associated protein. Such bispecific antibodies are capable of disrupting the suppression of anti-tumor activity by immune cells by targeting tumor-cell derived exosomes, which contain ligands, such as PD-L1, that inhibit T cell activation. Thus, compositions and methods of the invention can be used in the treatment of cancers.

The first exosome-associated target of a bispecific antibody may, for example, be a Tetraspanin transmembrane family protein, Tumor susceptibility gene 101 (“TSG101”), a Major histocompatibility complex (MHC) class II molecule, a Programmed cell death 6 interacting protein (“PDCD6IP”)′ a Heat shock protein, a cytoskeletal protein, an Annexin, or a membrane transport protein. Therefore, the first binding moiety of a bispecific antibody according to the invention can, for example, specifically bind Epsin-1 (“EPN1”), CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, and NKCC2.

The second binding moiety of a bispecific antibody, according to the invention, may be derived from any PD-L1-specific antibody, including the VH and a VL chains of a PD-L1-specific antibody, such as, but not limited to atezolizumab, avelumab, durvalumab, or BMS 936559.

### BRIEF DESCRIPTION OF THE FIGURES

FIG. 1 is a graphical representation of T cell co-stimulatory molecules of the B7 family.

FIG. 2. shows dose-dependent binding of exosomes to anti-CD63 coated beads as assayed by flow cytometry. Exosomes bound to the anti-CD63 beads were detected with fluorescently labeled anti-CD63 antibodies.

FIG. 3. shows that exosomes, captured on latex beads by adsorption, are reactive with the anti-EPN1 antibody IMM20059, when binding is assessed by flow cytometry. In contrast, IMM20059 does not bind to BSA-coated beads.

FIG. 4. shows the concentration-dependent binding curve observed for IMM20059 binding to intact A549 lung cancer cell lines by flow cytometry with an Attune™ N×T instrument (Life Technologies). Binding of IMM20059 to intact cells was detected with fluorophore-labelled anti-human secondary antibodies.

FIG. 5. shows the concentration-dependent binding curve observed for IMM20059 binding to intact Huh7 hepatocellular carcinoma cells by flow cytometry with an Attune™ N×T instrument (Life Technologies). Binding of IMM20059 to intact cells was detected with fluorophore-labelled anti-human secondary Abs.

FIG. 6. shows quantitative dot blot results depicting selectivity of IMM20059 for EPN1 over its homolog EPN2. Binding of IMM20059 was analyzed by dot blot against increasing concentrations of recombinant EPN1 or EPN2.

FIG. 7. shows a flow cytometry analysis demonstrating that IMM20059 cross-reacts with murine EPN1 antigen. Surface and intracellular staining of cells of the murine NIH3T3 and human MFE296 cell lines was performed. Cell surface and intracellular binding of IMM20059 was observed in pools of both cell lines. A commercially available anti-EPN1 antibody known to cross-react with both mouse and human EPN1 bound similarly to NIH3T3 and MFE296 cells. However, the commercial antibody failed to interact with EPN1 at the cell surface in both pools of cells.

FIG. 8. is a cartoon representation of two monospecific IgG antibodies and a bispecific antibody generated from the variable domains isolated from each of the two different IgG antibodies.

FIG. 9 is a cartoon representation depicting that bispecific anti-EPN1/anti-PDL1 antibodies will bind to exosomes containing both markers.

FIG. 10. shows dot blot results demonstrating that the position of the anti-PD-L1 variable domains within the bispecific antibody influences the ability of the antibody to bind PD-L1, but not EPN1.

FIG. 11. shows the concentration-dependent binding curve observed for the Ate/PR045-2H11:L anti-EPN1/anti-PD-L1 bispecific antibody to intact A549 lung cancer cell lines by flow cytometry with an Attune™ N×T instrument (Life Technologies). Binding of IMM20059 to intact cells was detected with fluorophore-labelled anti-human secondary antibodies.

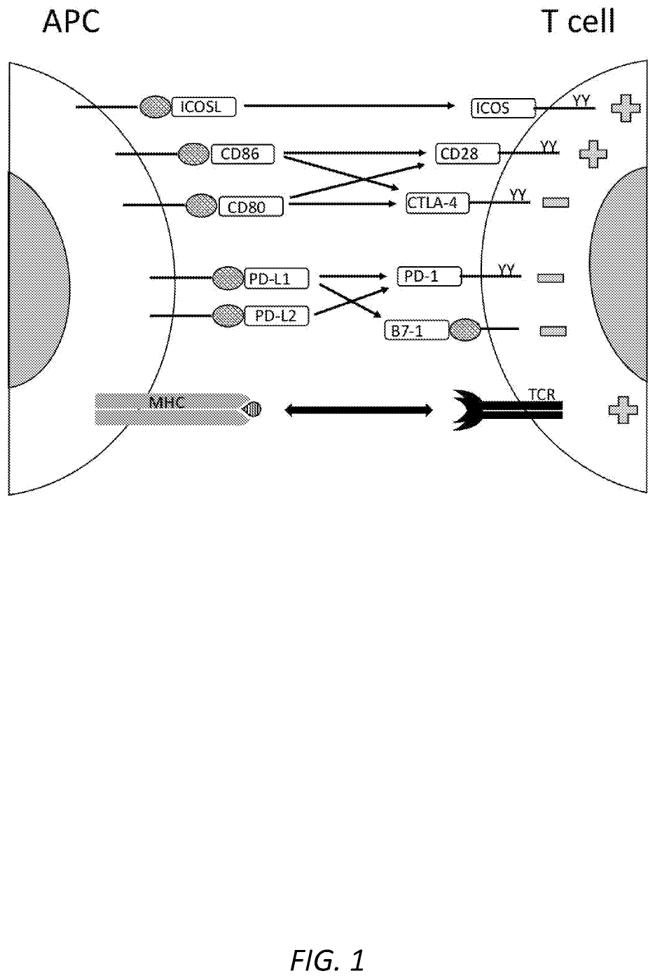

### DETAILED DESCRIPTION

The invention described herein is directed to bispecific antibodies that are capable of simultaneously targeting exosomes by specifically binding a first and second exosome-associated protein. More particularly, the second episome-associated protein, according to the invention, is Programmed Death Ligand-1 (“PD-L1”). Accordingly, a bispecific antibody according to the invention possesses a first antigen binding moiety that specifically binds an epitope on an exosomal-associated protein, and a second antigen binding moiety that specifically binds an epitope on PD-L1. Bispecific antibodies according to the invention can disrupt the suppression of anti-tumor activity by immune cells by targeting tumor-cell derived exosomes, which contain ligands, such as PD-L1, that inhibit T cell activation. Therefore, bispecific antibodies may be used to treat subjects afflicted by various types of cancers. Accordingly, the invention also includes compositions that are formulated for the administration and delivery of bispecific antibodies of the invention to subjects in need thereof, as a component of a cancer treatment protocol.

In general, exosomes are vesicles known to contain proteins belonging to one or more of the following groups: Tetraspanin transmembrane family proteins, such as CD9, CD63, and CD81; Tumor susceptibility gene 101 (“TSG101”); Major histocompatibility complex (MHC) class II molecules; Programmed cell death 6-interacting proteins (“PDCD6IPs”) 18,22,37,38,41; Heat shock proteins (HSP60, HSP70, and HSP90); Cytoskeletal proteins (actin and tubulin); Annexins (protein that regulate cytoskeletal changes in membranes and membrane fusion); and Membrane transport proteins. Exosomes are generally thought not to contain endoplasmic reticulum proteins, such as, calnexin and Golgi matrix proteins or nuclear proteins. It is known that exosomes can also contain the proteins CD10, CD26, CD37, CD45/ICAM-1, CD63, CD69, CD81, EGFR, EGFRvIII, EpCAM, Flotillin-1, Glypican-1, HER2, HER3, or NKCC2.

A basic antibody structure includes two heavy (H) and two light (L) polypeptide chains, each of which, contains a constant region and a variable region, and are interconnected by disulfide bonds. In humans, there are two types of immunoglobulin light chains, which are termed lambda (“λ”) and kappa (“κ”), and five main immunoglobulin heavy chain classes, also known as isotypes, which determine functional activity of an antibody molecule: IgM, IgD, IgG, IgA and IgE. Together, a variable heavy (“V_{H}”) region and a variable light (“V_{L}”) region form a fragment variable “Fv” that is responsible for the specific binding of the antibody to its antigen. A full-length heavy chain also has three constant domains (CH1, CH2, CH3). The constant regions of the Abs may mediate the binding of the immunoglobulin to host tissues or factors, including various cells of the immune system (e.g., effector cells) and the first component (C1q) of the classical complement system.

V_{H} and V_{L} regions contain “framework” regions interrupted by three hypervariable regions, called complementarity-determining regions (“CDRs”). The CDRs are primarily responsible for binding to an epitope of an antigen. The sequences of the framework regions of different light or heavy chains are relatively conserved within a species, and serve to position and align the CDRs in three-dimensional space. The three CDRs of each chain are typically referred to as CDR1, CDR2, and CDR3, numbered sequentially starting from the N-terminus, and are often identified by the chain in which the particular CDR is located. Accordingly, heavy chain CDRs are designated H-CDR1, H-CDR2, and H-CDR3; likewise, light chain CDRs are designated L-CDR1, L-CDR2, and L-CDR3. An antigen-binding fragment, one constant and one variable domain of each of the heavy and the light chain is referred to as an Fab fragment. An F(ab)′₂ fragment contains two Fab fragments, and can be generated by cleaving an immunoglobulin molecule below its hinge region.

Bispecific antibodies are capable of simultaneous binding of two different epitopes. A bispecific antibody according to the invention can be in the form of any immunoglobulin or immunoglobulin-derived molecule, or complex of molecules, that accommodates, in the same molecule. In various embodiments, the first binding moiety of a bispecific antibody according to the invention may be selected from an antibody that binds an epitope on an exosome-associated protein, such as, but not limited to Epsin-1 (“EPN1”), CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, and NKCC2. For example, a bispecific antibody can include a first antigen binding moiety that specifically binds an epitope on human EPN1, such as a binding moiety described in International Patent Application No. PCT/US19/54259, which is incorporated by reference. In various embodiments, the EPN1-specific first binding moiety of a bispecific antibody according to the invention can include a variable heavy chain as depicted in SEQ ID NO: 2 or SEQ ID NO: 6, a variable light chain as depicted in SEQ ID NO: 4 or SEQ ID NO: 8. In other embodiments, the first antigen-binding moiety of a bispecific antibody according to the invention has: (1) at least one of (a) a heavy chain CDR1 containing the amino acid sequence of SEQ ID NO: 9, (b) a heavy chain CDR2 comprising the amino acid sequence of SEQ ID NO: 10, and (c) a heavy chain CDR3 comprising the amino acid sequence of SEQ ID NO:11; (2) at least one of (a) a light chain CDR1 comprising the amino acid sequence of SEQ ID NO: 12, (b) a light chain CDR2 comprising the amino acid sequence of SEQ ID NO: 13, and (c) a light chain CDR3 comprising the amino acid sequence of SEQ ID NO: 14.

The second binding moiety of a bispecific antibody according to the invention, may be derived from any PD-L1-specific antibody, including the V_{H} and a V_{L} chains of a PD-L1-specific antibody, such as, but not limited to atezolizumab, avelumab, durvalumab, or BMS-936559.

With the foregoing descriptions of bispecific antibodies in mind, an embodiment of a bispecific antibody according to the invention may possess an EPN1-specific first binding moiety that has a heavy chain CDR1 based on SEQ ID NO: 9, a heavy chain CDR2 based on SEQ ID NO: 10, and a heavy chain CDR3 based on SEQ ID NO:11 a light chain CDR1 based on SEQ ID NO: 12, a light chain CDR2 based on SEQ ID NO: 13, and a light chain CDR3 based on SEQ ID NO: 14, and a PD-L1-specific second binding moiety that has heavy and light chain CDRs derived from a PD-L1-specific antibody. Therefore, another embodiment of a bispecific antibody according to the invention, may possess an EPN1-specific first binding moiety that has a heavy chain CDR1 based on SEQ ID NO: 9, a heavy chain CDR2 based on SEQ ID NO: 10, and a heavy chain CDR3 based on SEQ ID NO:11 a light chain CDR1 based on SEQ ID NO: 12, a light chain CDR2 based on SEQ ID NO: 13, and a light chain CDR3 based on SEQ ID NO: 14, and a PD-L1-specific second binding moiety that has a heavy chain CDR1 based on SEQ ID NO: 17, a heavy chain CDR2 based on SEQ ID NO: 18, and a heavy chain CDR3 based on SEQ ID NO:19 a light chain CDR1 based on SEQ ID NO: 20, a light chain CDR2 based on SEQ ID NO: 21, and a light chain CDR3 based on SEQ ID NO: 22.

A bispecific anti-CD-63/anti-PD-L1 antibody is another embodiment of a bispecific antibody capable of selectively targeting the PD-L1-positive exosomal pool. Embodiments of anti-CD-63/anti-PD-L1 bispecific antibodies include, but are not limited to, antibodies having variable domains, or the CDRs present within the variable domains, of an anti-CD-63 antibody (SEQ ID NOS: 44 and 45) in combination with the variable domains, or the CDRs present within the variable domains, of one of the anti-PD-L1 antibodies: atezolizumab (SEQ ID NOS: 15 and 16); avelumab (SEQ ID NOS: 23 and 24); durvalumab (SEQ ID NOS: 25 and 26); or BMS-936559 (SEQ ID NOS: 27 and 28). A preferred embodiment is an anti-CD-63/anti-PD-L1 bispecific antibody comprising the V_{H} and V_{L} domains of an anti-CD-63 antibody (SEQ ID NOS: 44 and 45) and atezolizumab, engineered into a DVD-Ig format. Four different configurations of the anti-CD-63 and atezolizumab variable domains are possible using the linkers and orientations defined for the anti-EPN-1/anti-PD-L1 bispecific antibodies.

A bispecific anti-HER2/anti-PD-L1 antibody is yet another embodiment of a bispecific antibody capable of selectively targeting the PD-L1-positive exosomal pool. Embodiments of an anti-HER2/anti-PD-L1 bispecific antibodies include, but are not limited to, having variable domains, or the CDRs present within the variable domains, of the anti-HER2 antibody trastuzumab (SEQ ID NOS: 46 and 47) in combination with the variable domains, or the CDRs present within the variable domains, of one of the anti-PD-L1 antibodies: atezolizumab (SEQ ID NOS: 15 and 16); avelumab (SEQ ID NOS: 23 and 24); durvalumab (SEQ ID NOS: 25 and 26); or BMS-936559 (SEQ ID NOS: 27 and 28). A preferred embodiment is an anti-HER2/anti-PD-L1 bispecific antibody comprising the V_{H} and V_{L} domains of an anti-HER2 antibody (SEQ ID NOS: 46 and 47) and atezolizumab, engineered into a DVD-Ig format. Four different configurations of the anti-HER2 and atezolizumab variable domains are possible using the linkers and orientations defined for the anti-EPN-1/anti-PD-L1 bispecific antibodies.

A bispecific anti-EpCAM/anti-PD-L1 antibody is still another embodiment of a bispecific antibody capable of selectively targeting the PD-L1-positive exosomal pool. Embodiments of an anti-EpCAM/anti-PD-L1 bispecific having variable domains, or the CDRs present within the variable domains, of the anti-EpCAM antibody oportuzumab (SEQ ID NOS: 48 and 49) in combination with the variable domains, or the CDRs present within the variable domains, of one of the anti-PD-L1 antibodies: atezolizumab (SEQ ID NOS: 15 and 16); avelumab (SEQ ID NOS: 23 and 24); durvalumab (SEQ ID NOS: 25 and 26); or BMS-936559 (SEQ ID NOS: 27 and 28). A preferred embodiment is an anti-EpCAM/anti-PD-L1 bispecific antibody comprising the V_{H} and V_{L} domains of an anti-EpCAM antibody (SEQ ID NOS: 48 and 49) and atezolizumab, engineered into a DVD-Ig format. Four different configurations of the anti-EpCAM and atezolizumab variable domains are possible using the linkers and orientations defined for the anti-EPN-1/anti-PD-L1 bispecific antibodies.

A bispecific anti-HER3/anti-PD-L1 antibody is still yet another embodiment of a bispecific antibody capable of selectively targeting the PD-L1-positive exosomal pool. Embodiments of an anti-HER3/anti-PD-L1 bispecific having variable domains, or the CDRs present within the variable domains, of the anti-HER3 antibody (SEQ ID NOS: 50 and 51) in combination with the variable domains, or the CDRs present within the variable domains, of one of the anti-PD-L1 antibodies: atezolizumab (SEQ ID NOS: 15 and 16); avelumab (SEQ ID NOS: 23 and 24); durvalumab (SEQ ID NOS: 25 and 26); or BMS-936559 (SEQ ID NOS: 27 and 28). A preferred embodiment is an anti-HER3/anti-PD-L1 bispecific antibody comprising the V_{H} and V_{L} domains of an anti-HER3 antibody (SEQ ID NOS: 50 and 51) and atezolizumab, engineered into a DVD-Ig format. Four different configurations of the anti-HER3 and atezolizumab variable domains are possible using the linkers and orientations defined for the anti-EPN-1/anti-PD-L1 bispecific antibodies.

Bispecific antibodies according to the invention are fully human or humanized monoclonal antibodies. In other words, a bispecific antibody according to the invention may include framework regions and CDRs derived from one or more human immunoglobulins. Indeed, the framework regions may originate from one human antibody, and be engineered to include CDRs from a different human antibody. For example, an antibody according to the invention may possess: i) one or more CDRs derived from a human antibody that is specific for an exosomal protein target; ii) one or more CDRs derived from a human antibody that is specific for PD-L1; and framework regions derived from another human antibody.

A bispecific antibody according to the invention can be an antibody fragment variant. For example, fragment variants of a bispecific antibody according to the invention include bivalent F(ab)′₂ fragments, bi-valent single chain Fv proteins (“bi-scFv”), and bi-valent disulfide stabilized Fv proteins (“bi-dsFv”). An (Fab′)₂ fragment is a dimer of two Fab′ fragments, that can be obtained by treating whole antibody with the enzyme pepsin without subsequent reduction, so Fab′ monomers remain held together by two disulfide bonds. A single chain (“sc”) antibody, such as a bi-scFv fragment, is a genetically engineered molecule containing the V_{L} and V_{H} regions of the heavy and light chains of a first antibody, and the V_{L} and V_{H} regions of the heavy and light chains of a second antibody, all linked by one or more suitable polypeptide linkers, to produce a genetically fused single chain molecule. A bispecific antibody according to the invention may also be a dimer of two different scFV antibodies. Yet other examples of bispecific antibodies include tandem scFv (taFv or scFv2), diabody, dAb2NHH2, knob-into-holes derivatives, SEED-IgG, heteroFc-scFv, Fab-scFv, scFvJun/Fos, Fab′-Jun/Fos, tribody, DNL-F(ab)3, scFv3-CHI/CL, Fab-scFv2, IgG-scFab, IgG-scFv, scFv-IgG, scFv2-Fc, F(ab′)2-scFv2, scDB-Fc, scDb-CH3, Db-Fe, scFv2-H/L, DVD-Ig, tandem diabody (“TandAb”), scFv-dhIx-scFv, dAb2-IgG, dAb-IgG, dAb-Fc-dAb.

One of skill in the art will realize that conservative variants of bispecific antibodies can be produced. Such conservative variants will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. The following six groupings of amino acids are examples of amino acids that are considered to be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); and vi) Phenylalanine (F), Tyrosine (Y), and Tryptophan (W).

A bispecific antibody according to the invention may also include a “tagged” immunoglobulin CH3 domain to facilitate detection of the biologic against a background of endogenous antibodies. More particularly, a tagged CH3 domain is a heterogenous antibody epitope that has been incorporated into one or more of the AB, EF, or CD structural loops of a human IgG-derived CH3 domain. CH3 tags are preferably incorporated into the structural context of an IgG1 subclass antibody, other human IgG subclasses, including IgG2, IgG3, and IgG4, are also available according to the invention. Epitope-tagged CH3 domains, also referred to as “CH3 scaffolds” can be incorporated into any antibody of the invention having a heavy chain constant region, generally in the form of an immunoglobulin Fc portion. Examples of CH3 scaffold tags, and methods for incorporating them into antibodies are disclosed in PCT Patent Application No. PCT/US19/32780. Antibodies used to detect epitope tagged CH3 scaffolds are generally referred to herein as “detector antibodies”.

Therapeutic effectiveness of a bispecific antibody according to the invention correlates with its binding affinity for its target antigens. Binding affinity may be calculated by a modification of the Scatchard method described by Frankel et al., Mol. Immunol., 16:101-106, 1979. Alternatively, binding affinity may be measured by the dissociation rate of an antibody from its antigen. Various methods can be used to measure binding affinity, including, for example, surface plasmon resonance (SPR), competition radioimmunoassay, ELISA, and flow cytometry.

An antibody that “specifically binds” an antigen is an antibody that binds the antigen with high affinity and does not significantly bind other unrelated antigens. High affinity binding of an antibody to its antigen is mediated by the binding interaction of one or more of the antibody's CDRs to an epitope, also known as an antigenic determinant, of the antigen target. Epitopes are particular chemical groups or peptide sequences on a molecule that are antigenic, meaning they are capable of eliciting a specific immune response. An epitope that is specifically bound by an antibody according to the invention, may be, for example, contained within a protein expressed by cells of one or more types of cancer. In general, an antibody exhibits “high affinity binding” if its dissociation constant value (“K_{D}”) is 50 nM, or less. Therefore, a bispecific antibody according to the invention exhibits high affinity binding to its exosomal protein or PD-L1 binding targets, if the K_{D} between the antibody and at least one of the binding targets is 50 nM, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, or 1 nM or less.

High affinity binding of a bispecific antibody according to the invention can, for example, be described with respect to its binding to a cell that expresses PD-L1. More particularly, an antibody according to the invention exhibits high affinity binding to PD-L1-expressing cells if it exhibits a half maximal effective concentration (EC₅₀) value of 10 nM or less, 9 nM or less, 8 nM or less, 7 nM or less, 6 nM or less, 5 nM or less, 4 nM or less, 3 nM or less, 2 nM or less, or 1 nM or less. Similarly, in addition to binding PD-L1 with high affinity, the same antibody can also bind a different exosome-associated protein with high affinity, such as bind to TSG101, CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, or NKCC2. In various variants, for example, a bispecific antibody according to the invention, exhibits an EC₅₀ to: (i) EPN1-expressing exosomes or cells of 10 nM or less, 9 nM or less, 8 nM or less, 7 nM or less, 6 nM or less, 5 nM or less, 4 nM or less, 3 nM or less, 2 nM or less, or 1 nM or less; and to PD-L1-expressing episomes or cells of 10 nM or less, 9 nM or less, 8 nM or less, 7 nM or less, 6 nM or less, 5 nM or less, 4 nM or less, 3 nM or less, 2 nM or less, or 1 nM or less.

As stated above, bispecific antibodies according to the invention can be used in methods for preventing, treating, or ameliorating a disease in a subject. More particularly, bispecific antibodies according to the invention can be used for preventing, treating, or ameliorating cancer. “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number of size of metastases. “Ameliorating” refers to the reduction in the number or severity of signs or symptoms of a disease, such as cancer. The amount of a bispecific antibody according to the invention, which provides either subjective relief of a symptom(s) or an objectively identifiable improvement as noted by a clinician or other qualified professional. A method for preventing, treating, or ameliorating cancer may require the administration of a composition, comprising an effective amount of a bispecific antibody according to the invention, to a subject to inhibit tumor growth or metastasis by disrupting the suppression of anti-tumor activity by immune cells by targeting tumor-cell derived exosomes that contain: i) PD-L1, which is a suppressor of anti-tumor-induced T cell activation; and ii) One other exosomal protein, which may, or may not, also suppress T cell activation. Therefore, administered bispecific antibody contacts tumor cell-derived exosomes, (i.e., is placed in direct physical association with the exosomes), where the bispecific antibody can bind at least one of its exosomal targets to prevent PD-L1 from functioning as a suppressor of T cell activation. In various embodiments, a bispecific antibody according to the invention prevents PD-L1-mediated cell signaling, which would otherwise transmit an inhibitory signal that reduces the proliferation of antigen-specific T-cells in lymph nodes, while simultaneously reducing apoptosis in regulatory T cells (anti-inflammatory, suppressive T cells).

Bispecific antibodies according to the invention, which are administered to subjects in need thereof, are formulated into compositions. More particularly, the bispecific antibodies can be formulated for systemic administration, or local administration, such as intra-tumor administration. For example, a bispecific antibody according to the invention may be formulated for parenteral administration, such as intravenous administration. The compositions can be prepared in unit dosage forms for administration to a subject. The amount and timing of administration are at the discretion of the treating clinician to achieve the desired outcome. Administration of bispecific antibodies according to the invention can also be accompanied by administration of other anti-cancer agents or therapeutic treatments, such as surgical resection of a tumor. Any suitable anti-cancer agent can be administered in combination with the bispecific antibodies disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

Compositions for administration can include a solution of a bispecific antibody dissolved in a pharmaceutically acceptable carrier, such as an aqueous carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, or glycerol as a vehicle. For solid compositions, such as powder, pill, tablet, or capsule forms, conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. The foregoing carrier solutions are sterile and generally free of undesirable matter, and may be sterilized by conventional, well known sterilization techniques. The compositions may contain pharmaceutically acceptable auxiliary substances as required to approximate physiological conditions such as pH adjusting and buffering agents, and toxicity adjusting agents such as sodium acetate, sodium chloride, potassium chloride, calcium chloride, and sodium lactate. The concentration of antibody in these formulations can vary widely, and will be selected primarily based on fluid volumes, viscosities, body weight and the like in accordance with the particular mode of administration selected and the subject's needs.

Options for administering bispecific antibody compositions according to the invention include, but are not limited to, administration by slow infusion, or administration via an intravenous push or bolus. Prior to being administered, a bispecific antibody composition according to the invention may be provided in lyophilized form, and rehydrated in a sterile solution to a desired concentration before administration. The bispecific antibody solution may, for example, then be added to an infusion bag containing 0.9% sodium chloride, USP, and in some cases administered at a dosage of from 0.5 to 20 mg/kg of body weight. In one example of administration of an antibody composition according to the invention, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Bispecific antibody compositions according to the invention may also be controlled release formulations. Controlled release parenteral formulations, for example, can be made as implants, or oily injections. Particulate systems, including microspheres, microparticles, microcapsules, nanocapsules, nanospheres, and nanoparticles, may also be used to deliver bispecific antibody compositions according to the invention. Microcapsules, as referred to herein, contain a bispecific antibody according to the invention as a central core component. In microspheres, an antibody according to the invention is dispersed throughout the particle. Particles, microspheres, and microcapsules smaller than about 1 μm are generally referred to as nanoparticles, nanospheres, and nanocapsules, respectively.

A bispecific antibody composition according to the invention can also be packaged into a kit for treating a cancer in a subject. Such a kit includes any composition disclosed herein. The kits may also include suitable storage containers, such as, ampules, vials, and tubes, for each pharmaceutical composition and other included reagents, such as buffers and balanced salt solutions, for use in administering the compositions to subjects. The compositions and other reagents may be present in the kits in any convenient form, such as, in a solution or in a powder form. The kits may further include instructions for use of the compositions. The kits may further include a packaging container, which may have one or more partitions for housing the pharmaceutical composition and other reagents.

Methods for making bispecific antibodies are known in the art. For example, bispecific antibodies can be produced recombinantly using the co-expression of two immunoglobulin heavy chain/light chain pairs. See, e.g., Milstein, et al. (1983) Nature 305: 537-39. Alternatively, bispecific antibodies can be prepared using chemical linkage. See, e.g., Brennan, et al. (1985) Science 229:81. Bispecific antibodies include bispecific antibody fragments. See, e.g., Bolliger, et al. (1993) Proc. Natl. Acad. Sci. U.S.A. 90:6444-48, Gruber, et al. (1994) J. Immunol. 152:5368. Accordingly, bispecific antibodies according to the invention can be produced by the expression of nucleic acid sequences encoding their amino acid sequences in living cells in culture. An “isolated” bispecific antibody according to the invention is one which has been substantially separated or purified away from other biological components environment, such as a cell, proteins and organelles. For example, a bispecific antibody may be isolated if it is purified to: i) greater than 95%, 96%, 97%, 98%, or 99% by weight of protein as determined by the Lowry method, and alternatively, more than 99% by weight; ii) a degree sufficient to obtain at least 15 residues of N-terminal or internal amino acid sequence by use of a spinning cup sequenator; iii) homogeneity by SDS-PAGE, under reducing or nonreducing conditions, using Coomassie blue or silver stain. Isolated antibody may also be an antibody according to the invention that is in situ within recombinant cells, since at least one component of the antibody's natural environment will not be present. Ordinarily, however, isolated antibody will be prepared by at least one purification step.

A variety of host-expression vector systems may be utilized to express a bispecific antibody according to the invention, by transforming or transfecting the cells with an appropriate nucleotide coding sequences for an antibody according to the invention. Examples of host-expression cells include, but are not limited to: Bacteria, such as E. coli and B. Subtilis, which may be transfected with bispecific antibody coding sequences contained within recombinant bacteriophage DNA, plasmid DNA, or cosmid DNA expression vectors; Yeast, such as Saccharomyces and Pichia, transformed with recombinant yeast expression vectors containing antibody coding sequences; Insect cell systems, infected with recombinant virons expression vectors, such as baculovirus, containing antibody coding sequences; Plant cell systems infected with recombinant vims expression vectors, such as cauliflower mosaic virus (“CaMV”), or tobacco mosaic vims (“TMV”), containing antibody coding sequences; and Mammalian cell systems, such as, but not limited to COS, Chinese hamster ovary (“CHO”) cells, ExpiCHO, baby hamster kidney (“BHK”) cells, HEK293, Expi293, 3T3, NSO cells, harboring recombinant expression constructs containing promoters derived from the genome of mammalian cell, such as the metallothionein promoter or elongation factor I alpha promoter, or from mammalian viruses, such as the adenovirus late promoter, and the vaccinia virus 7.5K promoter. For example, mammalian cells such as Human Embryonic Kidney 293 (HEK293) or a derivative thereof, such as Expi293, in conjunction with a dual promoter vector that incorporates mouse and rat elongation factor 1 alpha promoters to express the heavy and light chain fragments, respectively, is an effective expression system for antibodies according to the invention, which can be advantageously selected, depending upon the use intended for the antibody molecule being expressed.

When a large quantity of a bispecific antibody according to the invention is to be produced for the generation of a pharmaceutical composition of the antibody, vectors which direct the expression of high levels of readily purified fusion protein products may be desirable. Such vectors include, but are not limited to: a pUR278 vector (Ruther et al. EMBO J. 2:1791 (1983)), in which the antibody coding sequence may be ligated individually into the vector in frame with a lac Z coding region so that a fusion protein is produced; a pIN vector (Inouye & Inouye, Nucleic Acids Res. 13:3101-3109 (1985), and Van Heeke & Schuster, J. Biol. Chem. 24:5503-5509 (1989)); a pGEX vectors to fuse antibodies of the invention with glutathione S-transferase (“GST”). A GST fusion protein of an antibody according to the invention and a polypeptide tag is soluble and can easily be purified from lysed cells, by adsorption and binding to matrix glutathione-agarose beads, followed by elution in the presence of free glutathione. The pGEX vectors, by contrast, are designed to include thrombin or factor Xa protease cleavage sites so that the cloned target gene product—an antibody according to the invention—can be released from the GST moiety.

A host expression cell system may also be chosen which modulates the expression of inserted sequence(s) coding for an antibody according to the invention, or modifies and processes the gene product as desired. For example, modifications, including the glycosylation and processing, such as cleavage of protein products, may be important for the function of the protein. Indeed, different host cells have characteristic and specific mechanisms for the posttranslational processing and modification of proteins and gene products. To this end, eukaryotic host cells, which possess appropriate cellular machinery for proper processing of a primary transcript, as well as the glycosylation and phosphorylation of a gene product according to the invention may be used.

### Examples

The following Examples describe the design and characterization of bispecific antibodies targeting exosomes.

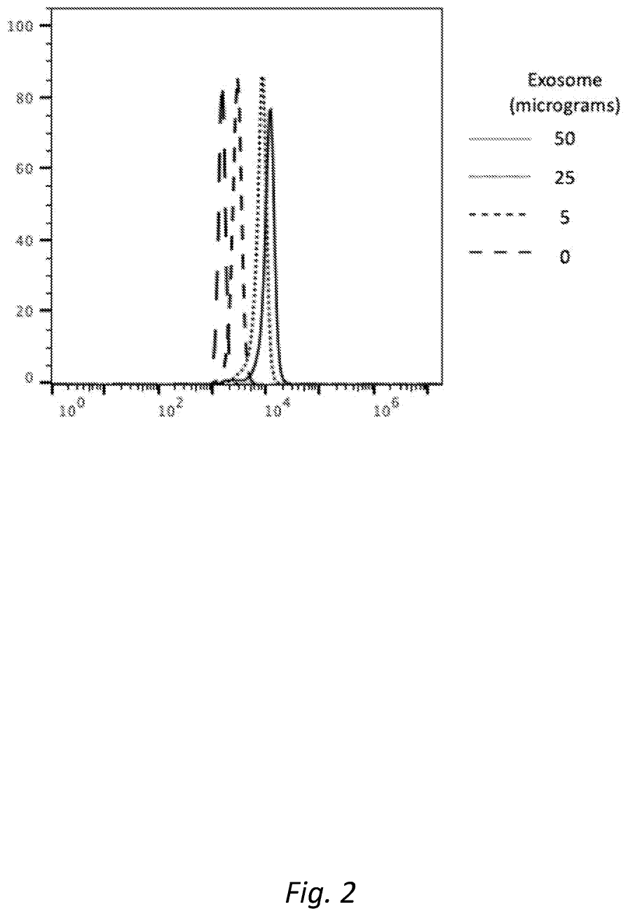

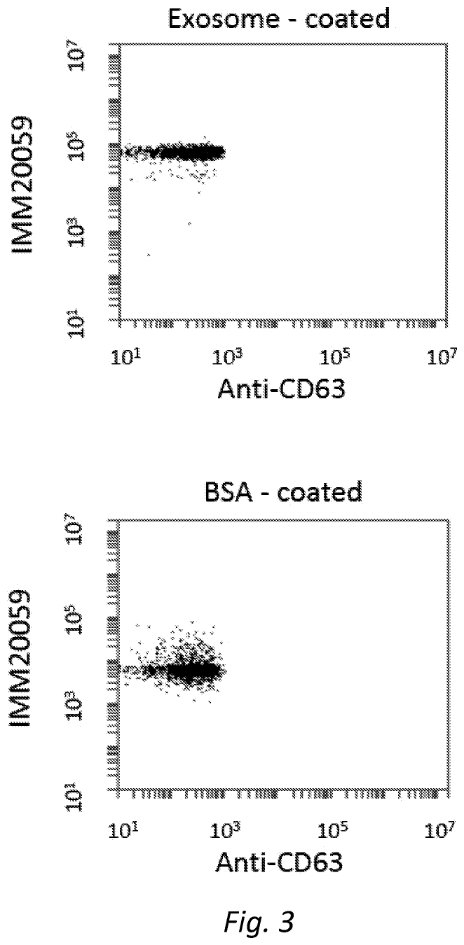

Example 1. Exosomes contain membrane bound proteins that can be targets for antibodies. Cells, derived from both normal and tumor tissues, can generate at least two classes of extracellular vesicles (EV), exosomes and ectosomes, which are derived through distinct biological processes. EVs are recognized to play a role in cellular communication. EVs are characterized by a series of different protein constituents, including proteins that are inserted into the lipid bilayer of the vesicles. Proteins known to be present in exosomal membranes can be divided into functional classes that include, but are not limited to, tetraspanins, heat shock proteins, membrane transporters, cell surface receptors, and lipid-bound molecules. Recognized proteins comprising those functional classes include, but are not limited to, TSG101, CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, NKCC2, and PD-L1. Proteins present on the surface of exosomes, such as CD63, can be detected by antibodies specific for those surface molecules. FIG. 2 demonstrates that exosomes derived from 22Rv1 prostate cancer cells can be isolated, in a dose-dependent manner, through interaction with anti-CD63 coated beads. The composition of transmembrane proteins associated with exosomes can be dependent upon cell type from which the exosomes are derived. Bulk preparations of exosomes can be conjugated to latex beads and detected with anti-CD63 antibodies by flow cytometry (FIG. 3). Bulk exosome-coated beads were also reactive with the anti-EPN1 antibody IMM20059. The IMM20059 staining was dependent upon exosomes being present on the bead surface; BSA-coated beads failed to interact with IMM20059. Data suggest that EPN1 is present on the surface of, at least a portion of, exosomes.

Example 2. IMM20059 is an antibody that binds to EPN1. The human hybridoma PR045-2H11 was created by created by fusing human B cells, isolated from the lymph node of a head and neck cancer patient, with the B56T fusion partner. Fusion of human B cells with B56T was carried out by electrofusion essentially as described in USPTO #EP2242836 “Method of making hybrid cells that express useful antibodies.” Nucleotide sequences, encoding the variable heavy chain (V_{H}) and variable light chain (V_{L}) domains of PR045-2H11, were obtained by RT-PCR amplification of RNA isolated from cells of the hybridoma line that produced PR045-2H11, and subjecting the resulting antibody cDNA to sequencing reactions. SEQ ID NO: 1 corresponds to the V_{H} and SEQ ID NO: 3 corresponds to the V_{L} of PR045-2H11 isolated from the hybridoma. Due to the RT-PCR strategy these sequences lack regions corresponding to the 5′ most portion of framework 1 of the variable domains. IGHV and IGKL gene assignments were predicted based upon homology to known germline gene sequences, and used as surrogates for the bona fide 5′ ends of the V_{H} and V_{L} sequences. IMM20059 is a recombinantly expressed human IgG1 antibody comprising the PR045-2H11 V_{H} and V_{L} domains. An expression fragment for IMM20059 V_{H} (SEQ ID NO: 5) was generated using germline sequence corresponding to 5′ end of framework 1 of IGHV3-48*02. A full-length expression fragment for PR045-2H11 V_{L} (SEQ ID NO: 7) was generated using the germline sequence corresponding to the 5′ end of framework 1 of IGKV3-11*01. Fragments corresponding to SEQ ID NO: 5 and SEQ ID NO: 7, were synthesized with additional 5′ and 3′ extensions to facilitate Gibson-style cloning into a dual promoter IgG1 expression vector. The corresponding protein sequences encoded by the V_{H} and V_{L} fragments are defined in SEQ ID NO: 6 and SEQ ID NO: 8, respectively. The coding region of the V_{H} and V_{L} domains have the hallmarks of somatic hypermutation, differing from germline sequences by 15 and 14 nucleotides respectively.

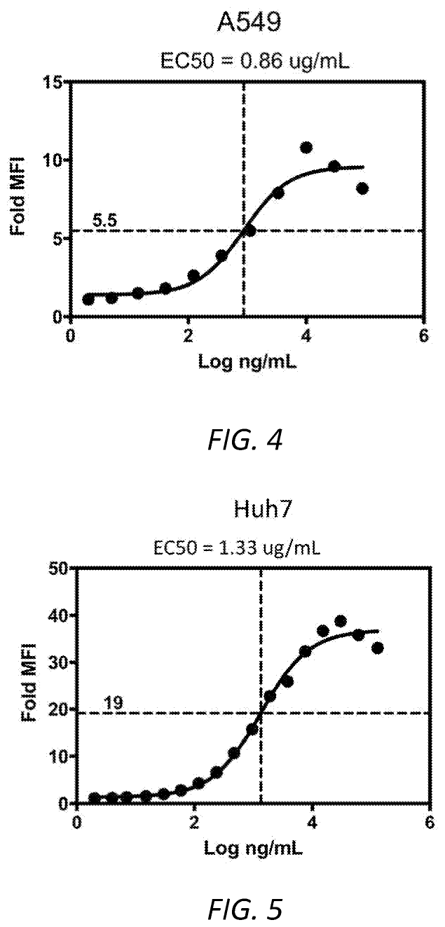

IMM20059 was expressed recombinantly by transient transfection into Expi293 cells using manufacturer recommended conditions. Recombinant antibody was purified from conditioned media by Protein A/G affinity chromatography, buffer exchanged into PBS and analyzed for activity by flow cytometry. IMM20059 displays binding activity consistent with the original PR045-2H11 hybridoma-produced antibody. As depicted in FIGS. 4 and 5, IMM20059 displays saturable binding to the surface of A549 lung adenocarcinoma and Huh7 hepatocellular carcinoma cell lines when analyzed by flow cytometry. IMM20059 binds to A549 and Huh7 with an EC50 of 0.9 and 1.3 μg/mL, respectively. These values correspond to EC50 values of between 6-9 nM.

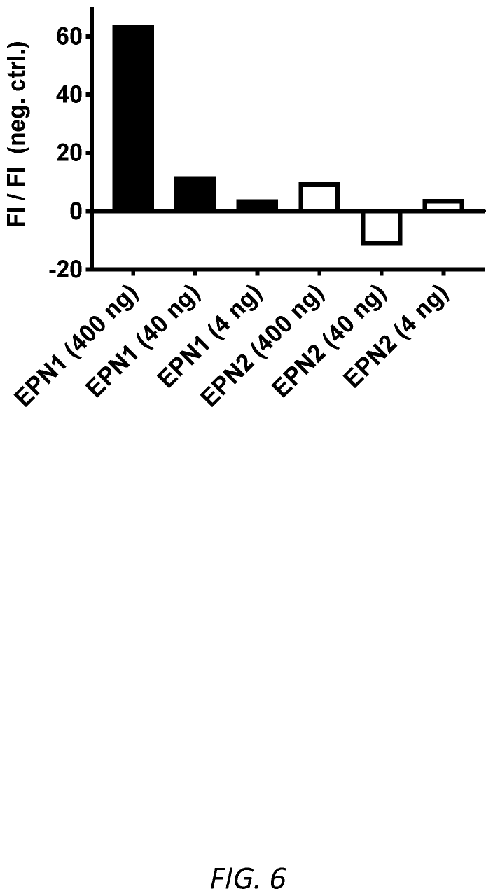

IMM20059 binds selectively, in a dose-dependent manner, to recombinant EPN1 as compared to its homolog EPN2 (FIG. 6). IMM20059 also displayed selectivity for EPN1 as compared to EPN3 in an reverse phase protein assay (RPPA). The strength of the interaction with recombinant EPN1 was further defined by surface plasmon resonance (Table 1). IMM20059, or an isotype control, were captured on an anti-human Fc sensor surface to generate binding and control surfaces. Recombinant EPN1 was flowed over the surfaces at increasing concentrations, in triplicate. Double-subtracted data was fit to a 1:1 binding model. As outlined in Table 1, IMM20059 demonstrated reproducible binding to EPN1 with an average K_{D} of 950+/−10 pM.

TABLE 1
Binding parameters determined for IMM20059/EPN1 at 25° C.

| Test | kₐ (M⁻¹s⁻¹) | k_{d} (s⁻¹) | K_{D} (pM) |
|---|---|---|---|
| 1^{st} | 7.3(2)e5 | 7.05(7)e−1 | 960(20) |
| 2^{nd} | 7.87(7)e5 | 7.36(7)e−4 | 940(10) |
| 3^{rd} | 7.6(1)e5 | 7.21(8)e−4 | 950(10) |
| Average | 7.6[3]e5 | 7.2[2]e−4 | 950[10] |

The numbers in parentheses are the errors in the last digits for the fits determined in the individual tests. The numbers in brackets are the experimental errors determined across the three tests.

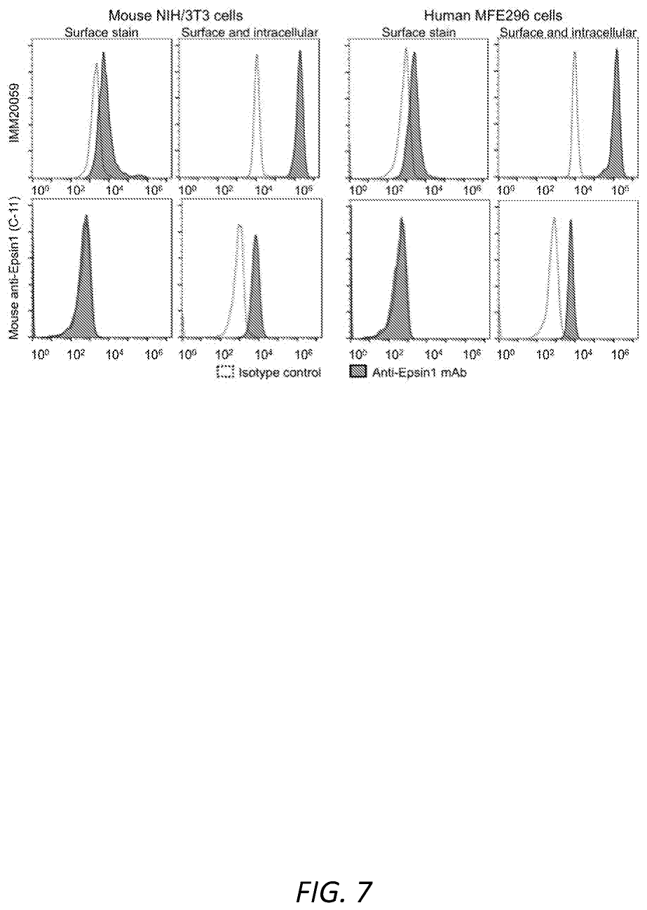

IMM20059 binds to the surface of EPN-1 positive murine cells. As depicted in FIG. 7, IMM20059 binds to both the cell surface and intracellular pools of antigen present in the murine NIH-3T3 cells. This pattern of binding is also observed against the human cell line MFE296. Commercially available anti-murine EPN1 antibodies do not recognize the cell surface pool of EPN1.

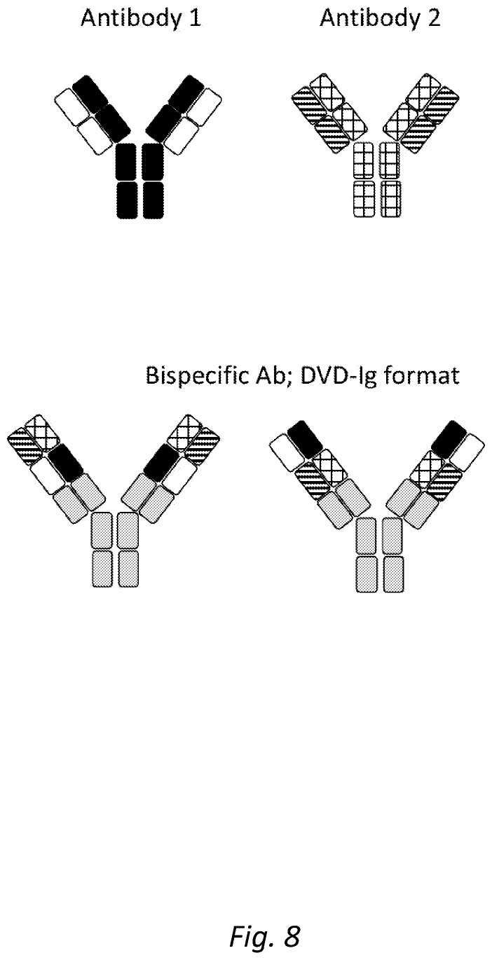

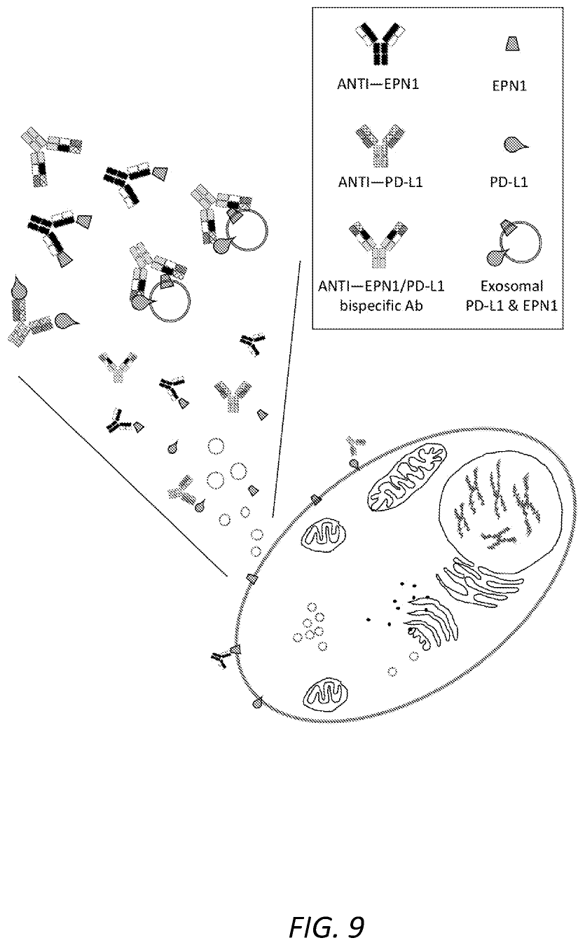

Example 3. Design of an anti-EPN-1/anti-PD-L1 bispecific antibody. Bispecific antibodies, antibodies capable of binding to two unique target antigens, can be created by combining variable domains from two mono-specific antibodies into one antibody-like molecule. Multiple bispecific antibody structures have been described in the literature (Brinkman, U. and Kontermann, R. mAbs, 9:182-212; 2017). One embodiment of a bispecific antibody structure is the dual variable domain—Ig (DVD-Ig). FIG. 8 is a cartoon representation of two monospecific antibodies and a DVD-Ig format bispecific antibody generated from the two monospecific antibodies. Bispecific antibodies are capable of improving targeting selectivity to cells, and by extension to exosomes, that express both target antigens as compared to those that express only one of the targets (Robinson et al BR J Cancer 99: 1415-1425; 2008). FIG. 9 is a cartoon representation of exosomal targeting by a bispecific antibody, capable of binding to both EPN1 and PD-L1, as compared to mono-specific antibodies capable of targeting only EPN-1 or PD-L1.

A number of anti-PD-L1 antibodies are described in the literature. They include, but are not limited to, atezolizumab, avelumab, durvalumab, and BMS-936559. A bispecific antibody capable of co-targeting exosomal PD-L1 and a second exosomal marker, could be developed to selectively target exosomal PD-L1 as compared to tumor cell localized PD-L1. Exosomal markers that could be targeted in a PD-L1 bispecific include, but are not limited to, CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, NKCC2 and EPN-1. A bispecific anti-EPN-1/anti-PD-L1 antibody represents one possible embodiment. A preferred embodiment is an anti-EPN-1/anti-PD-L1 bispecific comprising the variable domains, or the CDRs present within the variable domains, of IMM20059 in combination with the variable domains, or the CDRs present within the variable domains, of one of the anti-PD-L1 antibodies atezolizumab (SEQ ID NOS: 15 and 16), avelumab (SEQ ID NOS: 23 and 24), durvalumab (SEQ ID NOS: 25 and 26), or BMS-936559 (SEQ ID NOS: 27 and 28). A preferred embodiment is an anti-EPN-1/anti-PD-L1 bispecific antibody comprising the V_{H} and V_{L} domains of IMM20059 and atezolizumab, engineered into a DVD-Ig format. Four different configurations of the IMM20059 and atezolizumab variable domains were designed. The V_{H} domains were linked via the peptide linker ASTKGPSVFPLAP (SEQ ID NO: 29) in both an IMM20059-L-atezolizumab (SEQ ID NO: 33) orientation and atezolizumab-L-IMM20059 (SEQ ID NO: 39). The V_{L} domains of IMM20059 and atezolizumab were fused into a single polypeptide with two different linkers and in both orders from N- to C-terminus. The “L” linker comprises the amino acid sequence TVAAPSVFIFPP (SEQ ID NO: 30) and the “S” linker comprises the amino acid sequence TVAAP (SEQ ID NO: 31). SEQ ID NO: 35 and SEQ ID NO: 41 represent the “L” linker containing constructs in the IMM20059-L-atezolizumab and atezolizumab-L-IMM20059 orders, respectively. SEQ ID NO: 37 and SEQ ID NO: 43 correspond to the bispecific constructs linked by the “S” linker sequence.

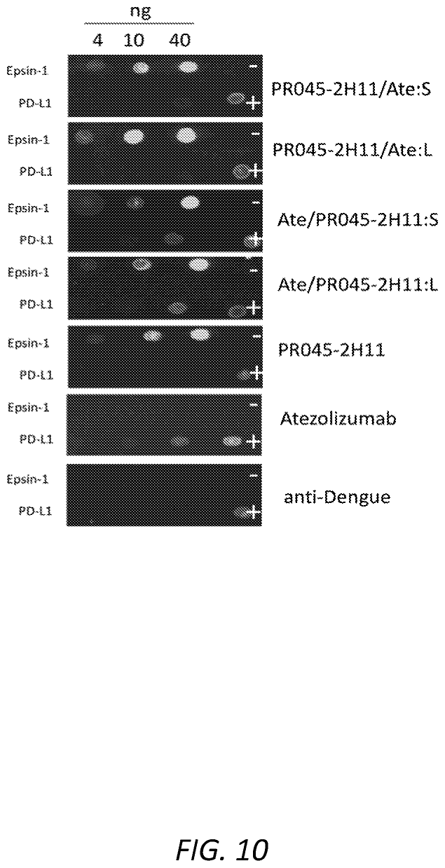

Example 4. Binding activity of anti-EPN1/anti-PD-L1 DVD-IgG bispecific antibodies. Four anti-EPN1/anti-PD-L1 bispecific antibodies were purified, by protein A affinity chromatography, from the conditioned media of a derivative of the HEK293 mammalian cell line that had been transiently transfected with plasmids encoding the heavy and light chains of a bispecific antibody. The amino acid sequences of the variable heavy and variable light domains comprising the four bispecific antibodies were SEQ ID NOS: 33 and 35, SEQ ID NOS: 33 and 37, SEQ ID NOS: 39 and 41, and SEQ D NOS: 39 and 43. Purified antibodies were subjected to dot blot analysis to determine if they were capable of binding to both recombinant EPN1 and recombinant PD-L1. Purified recombinant proteins were spotted at three dose levels as depicted in FIG. 10, and probed with the four anti-EPN1/anti-PD-L1 bispecific antibodies. Monospecific IMM20059/PR045-2H11 and atezolizumab served as positive controls for binding to EPN1 and PD-L1, respectively. An antibody specific for a coat protein on the dengue virus served as a negative control. All four bispecific antibodies bound to EPN1 to similar levels as IMM20059. Binding to PD-L1 required that the anti-PD-L1 variable domains be present at the N-terminus of the DVD-IgG (Ate/PR045-2H11:S and Ate/PR045-2H11:L). Positioning them C-terminal to the anti-EPN1 variable domain (PR045-2H11/Ate:S and PR045-2H11/Ate:L), diminished the ability to bind to PD-L1 in the dot blot format. The length of the linker within the variable light construct did not impact binding. Antibodies containing variable light domains corresponding to SEQ ID NOS: 37 and 39 bound equivalently to recombinant PD-L1 in the dotblot format.

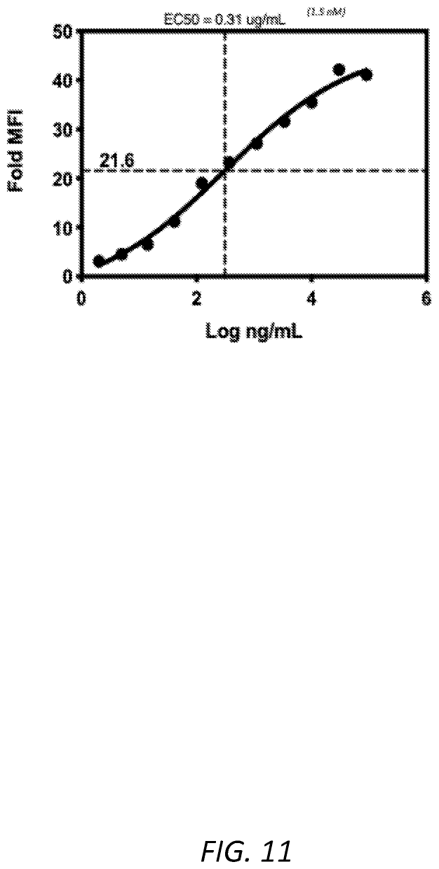

When analyzed by flow cytometry, the bispecific antibody comprising the variable domains defined by SEQ ID NOS: 33 and 39 bound to the surface of A549 cells, which are known to express both EPN1 and PD-L1 on the cell surface. Binding of the bispecific antibody to the cell surface exhibited a dose-dependent binding profile with an EC50 of approximately 0.3 microgram/mL (FIG. 11)

SEQUENCE LISTINGS

SEQ ID NO: 1-V_{H} PR045-2H11 nucleotide sequence
GACTCTCCTGTGCAGCCTCTGGATTCACCTTCAGTATCCATAGCCTGAATTGGGTCCGCCAGGCTCCAGGGAAGGG
ACTGGAGTGGGTTTCGTATATTAGTAGTAACAGTACTACCATATATTACGCAGACTCTGTGAAGGGCCGATTCACC
ATCTCCAGAGACAATGCCAAGGACTCCCTGTATCTGCAAATGAACAGCCTCAGAGACGAGGACACGGCTGTATAT
TACTGTGCGAGAGACTACTACTGTACTGGTGGTACCTGCTTCTTTCTTCCTGACCTCTGGGGCCGGGGAGCCCTGG
TCACCGTCTCCTCAGCCTCCACCAAGGGCCCATCGGTCTTCCCCCTGGCGC
SEQ ID NO: 2-V_{H} PR045-2H11 amino acid sequence
LSCAASGFTFSIHSLNWVRQAPGKGLEWVSYISSNSTTIYYADSVKGRFTISRDNAKDSLYLQMNSLRDEDTAVYYCARD
YYCTGGTCFFLPDLWGRGALVTVSSASTKKGPSVFPLA
SEQ ID NO: 3-V_{L} PR045-2H11 nucleotide sequence
AAGAGCCACCCTCTCCTGCAGGGCCAGTCAGAATATCAGCAACTTCTTAGCCTGGTACCAACACAAACCTGGCCAG
GCTCCCAGGCTCCTCATCTATGATGCATCCATCAGGGCCACTGGCATCCCAGCCAGGTTCAGTGGCAGTGGGTCTG
GGACAGACTTCAGTCTCACCATCAGCAGCCTGGAGCCTGAAGATTTTGCAGTTTATTTCTGTCAGCAGCGTTACAA
CTGGCTCACTTTCGGCGGAGGGACCAAGGTAGAGATCAAACGAACTGTGGCTGCACCATCTGTCTTCATCT
SEQ ID NO: 4-V_{L} PR045-2H11 amino acid sequence
RATLSCRASQNISNFLAWYQHKPGQAPRLLIYDASIRATGIPARFSGSGSGTDFSLTISSLEPEDFAVYFCQQRYNWLTFG
GGTKVEIKRTVAAPSVFI
SEQ ID NO: 5-IMM20059 V_{H} domain nucleotide sequence
ACAGGCGCGCACTCCGAGGTGCAGCTGGTGGAGTCTGGGGGAGGCTTGGTACAGCCTGGGGGGTCCCTGAGACT
CTCCTGTGCAGCCTCTGGATTCACCTTCAGTATCCATAGCCTGAATTGGGTCCGCCAGGCTCCAGGGAAGGGACTG
GAGTGGGTTTCGTATATTAGTAGTAACAGTACTACCATATATTACGCAGACTCTGTGAAGGGCCGATTCACCATCT
CCAGAGACAATGCCAAGGACTCCCTGTATCTGCAAATGAACAGCCTCAGAGACGAGGACACGGCTGTATATTACT
GTGCGAGAGACTACTACTGTACTGGTGGTACCTGCTTCTTTCTTCCTGACCTCTGGGGCCGGGGAGCCCTGGTCAC
CGTCTCCTCAGCCTCCACCAAGGGCCCATC
SEQ ID NO: 6-IMM20059 V_{H} domain amino acid sequence
EVQLVESGGGLVQPGGSLRLSCAASGFTFSIHSLNWVRQAPGKGLEWVSYISSNSTTIYYADSVKGRFTISRDNAKDSLY
LQMNSLRDEDTAVYYCARDYYCTGGTCFFLPDLWGRGALVTVSSASTKGPSVFPL
SEQ ID NO: 7-IMM20059 V_{L} domain nucleotide sequence
TCAGATACCTCCGGAGAAATTGTGTTGACACAGTCTCCAGCCACCCTGTCTTTGTCTCCAGGGGAAAGAGCCACCC
TCTCCTGCAGGGCCAGTCAGAATATCAGCAACTTCTTAGCCTGGTACCAACACAAACCTGGCCAGGCTCCCAGGCT
CCTCATCTATGATGCATCCATCAGGGCCACTGGCATCCCAGCCAGGTTCAGTGGCAGTGGGTCTGGGACAGACTTC
AGTCTCACCATCAGCAGCCTGGAGCCTGAAGATTTTGCAGTTTATTTCTGTCAGCAGCGTTACAACTGGCTCACTTT
CGGCGGAGGGACCAAGGTAGAGATCAAACGAACTGTGGCTG
SEQ ID NO: 8-IMM20059 V_{L} domain amino acid sequence
EIVLTQSPATLSLSPGERATLSCRASQNISNFLAWYQHKPGQAPRLLIYDASIRATGIPARFSGSGSGTDFSLTISSLEPEDF
AVYFCQQRYNWLTFGGGTKVEIKRTVA
SEQ ID NO: 9-IMM20059 H-CDR1
SIHSLN
SEQ ID NO: 10-IMM20059 H-CDR2
YISSNSTTIYYADSVKG
SEQ ID NO: 11-IMM20059 H-CDR3
DYYCTGGTCFFLPDL
SEQ ID NO: 12-IMM20059 L-CDR1
RASQNISNFLA
SEQ ID NO: 13-IMM20059 L-CDR2
DASIRAT
SEQ ID NO: 14-IMM20059 L-CDR3
QQRYNWLT
SEQ ID NO: 15-atezolizumab V_{H} domain
EVQLVESGGGLVQPGGSLRLSCAASGFTFSDSWIHWVRQAPGKGLEWVAWISPYGGSTYYADSVKGRFTISADTSKNT
AYLQMNSLRAEDTAVYYCARRHWPGGFDYWGQGTLVTVSSASTKGPSVFPLA
SEQ ID NO: 16-atezolizumab V_{L} domain
DIQMTQSPSSLSASVGDRVTITCRASQDVSTAVAWYQQKPGKAPKLLIYSASFLYSGVPSRFSGSGSGTDFTLTISSLQPE
DFATYYCQQYLYHPATFGQGTKVEIKRTVA
SEQ ID NO: 17-atezolizumab H-CDR1
SDSWIH
SEQ ID NO: 18-atezolizumab H-CDR2
PYGGSTYYADSVKG
SEQ ID NO: 19-atezolizumab H-CDR3
ARRHWPGGFDY
SEQ ID NO: 20-atezolizumab L-CDR1
RASQDVSTAVA
SEQ ID NO: 21-atezolizumab L-CDR2
SASFLYS
SEQ ID NO: 22-atezolizumab L-CDR3
QQYLYHPAT
SEQ ID NO: 23-avelumab V_{H} domain
EVQLLESGGGLVQPGGSLRLSCAASGFTFSSYIMMWVRQAPGKGLEWVSSIYPSGGITFYADTVKGRFTISRDNSKNTL
YLQMNSLRAEDTAVYYCARIKLGTVTTVDYWGQGTLVTVSSASTKGPSVFPLA
SEQ ID NO: 24-avelumab V_{L} domain
QSALTQPASVSGSPGQSITISCTGTSSDVGGYNYVSWYQQHPGKAPKLMIYDVSNRPSGV
SNRFSGSKSGNTASLTISGLQAEDEADYYCSSYTSSSTRVFGTGTKVTVLG
SEQ ID NO: 25-durvalumab V_{H} domain
EVQLVESGGGLVQPGGSLRLSCAASGFTFSRYWMSWVRQAPGKGLEWVANIKQDGSEKYYVDSVKGRFTISRDNAK
NSLYLQMNSLRAEDTAVYYCAREGGWFGELAFDYWGQGTLVTVSSASTKGPSVFPLA
SEQ ID NO: 26 durvalumab V_{L} domain
EIVLTQSPGTLSLSPGERATLSCRASQRVSSSYLAWYQQKPGQAPRLLIYDASSRATGIPDRFSGSGSGTDFTLTISRLEPE
DFAVYYCQQYGSLPWTFGQGTKVEIKRTVA
SEQ ID NO: 27 BMS-936559 V_{H} domain
QVQLVQSGAEVKKPGSSVKVSCKTSGDTFSTYAISWVRQAPGQGLEWMGGIIPIFGKAHYAQKFQGRVTITADESTST
AYMELSSLRSEDTAVYFCARKFHFVSGSPFGMDVWGQGTTVTVSSASTKGPSVFPLA
SEQ ID NO: 28 BMS-936559 V_{L} domain
EIVLTQSPATLSLSPGERATLSCRASQSVSSYLAWYQQKPGQAPRLLIYDASNRATGIPARFSGSGSGTDFTLTISSLEP
EDFAVYYCQQRSNWPTFGQGTKVEIKRTVA
SEQ ID NO: 29-V_{H} ″L″ linker
ASTKGPSVFPLAP
SEQ ID NO: 30-V_{L} ″L″ linker
TVAAPSVFIFPP
SEQ ID NO: 31-V_{L} ″S″ LINKER
TVAAP
SEQ ID NO: 32-IMM20059-L-ATE bispecific V_{H} domain nucleotide sequence
GAGGTGCAGCTGGTGGAGTCTGGGGGAGGCTTGGTACAGCCTGGGGGGTCCCTGAGACTCTCCTGTGC
AGCCTCTGGATTCACCTTCAGTATCCATAGCCTGAATTGGGTCCGCCAGGCTCCAGGGAAGGGACTGGA
GTGGGTTTCGTATATTAGTAGTAACAGTACTACCATATATTACGCAGACTCTGTGAAGGGCCGATTCACC
ATCTCCAGAGACAATGCCAAGGACTCCCTGTATCTGCAAATGAACAGCCTCAGAGACGAGGACACGGCT
GTATATTACTGTGCGAGAGACTACTACTGTACTGGTGGTACCTGCTTCTTTCTTCCTGACCTCTGGGGCC
GGGGAGCCCTGGTCACCGTCTCCTCAGCGAGCACAAAAGGACCATCTGTATTTCCACTCGCCCCCGAAG
TACAGCTCGTAGAGTCCGGAGGAGGCCTGGTCCAACCTGGTGGTTCCCTTCGACTGTCATGTGCCGCGT
CTGGCTTCACTTTTTCCGATTCATGGATACACTGGGTGAGGCAAGCACCTGGCAAAGGTTTGGAATGGG
TGGCCTGGATCTCACCGTATGGGGGTAGTACTTATTATGCGGATTCAGTAAAGGGAAGATTTACCATTTC
AGCGGACACAAGTAAAAATACCGCCTATTTGCAGATGAACAGCCTGCGAGCGGAAGACACTGCTGTCTA
TTATTGTGCTAGACGCCACTGGCCTGGTGGTTTTGACTACTGGGGGCAGGGCACTTTGGTGACCGTTTCC
TCA
SEQ ID NO: 33 IMM20059-L-ATE bispecific V_{H} domain amino acid sequence
EVQLVESGGGLVQPGGSLRLSCAASGFTFSIHSLNWVRQAPGKGLEWVSYISSNSTTIYYADSVKGRFTISRDNAKDSLY
LQMNSLRDEDTAVYYCARDYYCTGGTCFFLPDLWGRGALVTVSSASTKGPSVFPLAPEVQLVESGGGLVQPGGSLRLS
CAASGFTFSDSWIHWVRQAPGKGLEWVAWISPYGGSTYYADSVKGRFTISADTSKNTAYLQMNSLRAEDTAVYYCAR
RHWPGGFDYWGQGTLVTVSS
SEQ ID NO: 34-IMM20059-L-ATE bispecific V_{L} domain nucleotide sequence
GAAATTGTGTTGACACAGTCTCCAGCCACCCTGTCTTTGTCTCCAGGGGAAAGAGCCACCCTCTCCTGCAGGGCCA
GTCAGAATATCAGCAACTTCTTAGCCTGGTACCAACACAAACCTGGCCAGGCTCCCAGGCTCCTCATCTATGATGC
ATCCATCAGGGCCACTGGCATCCCAGCCAGGTTCAGTGGCAGTGGGTCTGGGACAGACTTCAGTCTCACCATCAG
CAGCCTGGAGCCTGAAGATTTTGCAGTTTATTTCTGTCAGCAGCGTTACAACTGGCTCACTTTCGGCGGAGGGACC
AAGGTAGAGATCAAACGAACAGTAGCAGCTCCGTCAGTTTTTATTTTTCCTCCAGATATTCAGATGACCCAGTCCCC
GTCCTCTCTCTCCGCTAGTGTAGGTGATAGAGTGACAATAACATGCCGGGCCAGCCAGGATGTATCCACGGCGGT
CGCGTGGTACCAGCAGAAACCTGGGAAAGCCCCCAAACTGCTTATTTATAGCGCCAGCTTCTTGTACTCAGGAGTA
CCTAGCAGATTTAGCGGTTCAGGAAGTGGGACTGATTTTACACTCACTATATCTTCCCTGCAACCGGAGGATTTTG
CAACATATTATTGTCAACAATATCTCTACCATCCCGCGACATTCGGGCAGGGCACAAAAGTAGAGATCAAACGA
SEQ ID NO: 35-IMM20059-L-ATE bispecific V_{L} domain amino acid sequence
EIVLTQSPATLSLSPGERATLSCRASQNISNFLAWYQHKPGQAPRLLIYDASIRATGIPARFSGSGSGTDFSLTISSLEPEDF
AVYFCQQRYNWLTFGGGTKVEIKRTVAAPSVFIFPPDIQMTQSPSSLSASVGDRVTITCRASQDVSTAVAWYQQKPGK
APKLLIYSASFLYSGVPSRFSGSGSGTDFTLTISSLQPEDFATYYCQQYLYHPATFGQGTKVEIKR
SEQ ID NO: 36-2H11-S-ATE bispecific V_{L} domain nucleotide sequence
GAAATTGTGTTGACACAGTCTCCAGCCACCCTGTCTTTGTCTCCAGGGGAAAGAGCCACCCTCTCCTGCAGGGCCA
GTCAGAATATCAGCAACTTCTTAGCCTGGTACCAACACAAACCTGGCCAGGCTCCCAGGCTCCTCATCTATGATGC
ATCCATCAGGGCCACTGGCATCCCAGCCAGGTTCAGTGGCAGTGGGTCTGGGACAGACTTCAGTCTCACCATCAG
CAGCCTGGAGCCTGAAGATTTTGCAGTTTATTTCTGTCAGCAGCGTTACAACTGGCTCACTTTCGGCGGAGGGACC
AAGGTAGAGATCAAACGAACAGTAGCAGCTCCGGATATTCAGATGACCCAGTCCCCGTCCTCTCTCTCCGCTAGTG
TAGGTGATAGAGTGACAATAACATGCCGGGCCAGCCAGGATGTATCCACGGCGGTCGCGTGGTACCAGCAGAAA
CCTGGGAAAGCCCCCAAACTGCTTATTTATAGCGCCAGCTTCTTGTACTCAGGAGTACCTAGCAGATTTAGCGGTT
CAGGAAGTGGGACTGATTTTACACTCACTATATCTTCCCTGCAACCGGAGGATTTTGCAACATATTATTGTCAACAA
TATCTCTACCATCCCGCGACATTCGGGCAGGGCACAAAAGTAGAGATCAAACGA
SEQ ID NO: 37-2H11-S-ATE bispecific V_{L} domain amino acid sequence
EIVLTQSPATLSLSPGERATLSCRASQNISNFLAWYQHKPGQAPRLLIYDASIRATGIPARFSGSGSGTDFSLTISSLEPEDF
AVYFCQQRYNWLTFGGGTKVEIKRTVAAPDIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATFGQGTKVEIKR
SEQ ID NO: 38-ATE-L-2H11 bispecific V_{H} domain nucleotide sequence
GAAGTACAGCTCGTAGAGTCCGGAGGAGGCCTGGTCCAACCTGGTGGTTCCCTTCGACTGTCATGTGCCGCGTCT
GGCTTCACTTTTTCCGATTCATGGATACACTGGGTGAGGCAAGCACCTGGCAAAGGTTTGGAATGGGTGGCCTGG
ATCTCACCGTATGGGGGTAGTACTTATTATGCGGATTCAGTAAAGGGAAGATTTACCATTTCAGCGGACACAAGTA
AAAATACCGCCTATTTGCAGATGAACAGCCTGCGAGCGGAAGACACTGCTGTCTATTATTGTGCTAGACGCCACTG
GCCTGGTGGTTTTGACTACTGGGGGCAGGGCACTTTGGTGACCGTTTCCTCAGCCGCGAGCACAAAAGGACCATC
TGTATTTCCACTCGCCCCCGAGGTGCAGCTGGTGGAGTCTGGGGGAGGCTTGGTACAGCCTGGGGGGTCCCTGA
GACTCTCCTGTGCAGCCTCTGGATTCACCTTCAGTATCCATAGCCTGAATTGGGTCCGCCAGGCTCCAGGGAAGGG
ACTGGAGTGGGTTTCGTATATTAGTAGTAACAGTACTACCATATATTACGCAGACTCTGTGAAGGGCCGATTCACC
ATCTCCAGAGACAATGCCAAGGACTCCCTGTATCTGCAAATGAACAGCCTCAGAGACGAGGACACGGCTGTATAT
TACTGTGCGAGAGACTACTACTGTACTGGTGGTACCTGCTTCTTTCTTCCTGACCTCTGGGGCCGGGGAGCCCTGG
TCACCGTCTCCTCAGCCTCCACCAAGGGCCCATCGGTC
SEQ ID NO: 39-ATE-L-2H11 bispecific V_{H} domain amino acid sequence
EVQLVESGGGLVQPGGSLRLSCAASGFTFSDSWIHWVRQAPGKGLEWVAWISPYGGSTYYADSVKGRFTISADTSKNT
AYLQMNSLRAEDTAVYYCARRHWPGGFDYWGQGTLVTVSSAASTKGPSVFPLAPEVQLVESGGGLVQPGGSLRLSCA
ASGFTFSIHSLNWVRQAPGKGLEWVSYISSNSTTIYYADSVKGRFTISRDNAKDSLYLQMNSLRDEDTAVYYCARDYYCT
GGTCFFLPDLWGRGALVTVSSASTKGPSV
SEQ ID NO: 40-ATE-L-2H11 bispecific V_{L} domain nucleotide sequence
GATATTCAGATGACCCAGTCCCCGTCCTCTCTCTCCGCTAGTGTAGGTGATAGAGTGACAATAACATGCCGGGCCA
GCCAGGATGTATCCACGGCGGTCGCGTGGTACCAGCAGAAACCTGGGAAAGCCCCCAAACTGCTTATTTATAGCG
CCAGCTTCTTGTACTCAGGAGTACCTAGCAGATTTAGCGGTTCAGGAAGTGGGACTGATTTTACACTCACTATATCT
TCCCTGCAACCGGAGGATTTTGCAACATATTATTGTCAACAATATCTCTACCATCCCGCGACATTCGGGCAGGGCA
CAAAAGTAGAGATCAAACGAACCGTCGCCGCACCATCAGTTTTTATTTTTCCTCCAGAAATTGTGTTGACACAGTCT
CCAGCCACCCTGTCTTTGTCTCCAGGGGAAAGAGCCACCCTCTCCTGCAGGGCCAGTCAGAATATCAGCAACTTCT
TAGCCTGGTACCAACACAAACCTGGCCAGGCTCCCAGGCTCCTCATCTATGATGCATCCATCAGGGCCACTGGCAT
CCCAGCCAGGTTCAGTGGCAGTGGGTCTGGGACAGACTTCAGTCTCACCATCAGCAGCCTGGAGCCTGAAGATTT
TGCAGTTTATTTCTGTCAGCAGCGTTACAACTGGCTCACTTTCGGCGGAGGGACCAAGGTAGAGATCAAACGA
SEQ ID NO: 41-ATE-L-2H11 bispecific V_{L} domain amino acid sequence
DIQMTQSPSSLSASVGDRVTITCRASQDVSTAVAWYQQKPGKAPKLLIYSASFLYSGVPSRFSGSGSGTDFTLTISSLQPE
DFATYYCQQYLYHPATFGQGTKVEIKRTVAAPSVFIFPPEIVLTQSPATLSLSPGERATLSCRASQNISNFLAWYQHKPGQ
APRLLIYDASIRATGIPARFSGSGSGTDFSLTISSLEPEDFAVYFCQQRYNWLTFGGGTKVEIKR
SEQ ID NO: 42-ATE-S-2H11 bispecific V_{L} domain nucleotide sequence
GATATTCAGATGACCCAGTCCCCGTCCTCTCTCTCCGCTAGTGTAGGTGATAGAGTGACAATAACATGCCGGGCCA
GCCAGGATGTATCCACGGCGGTCGCGTGGTACCAGCAGAAACCTGGGAAAGCCCCCAAACTGCTTATTTATAGCG
CCAGCTTCTTGTACTCAGGAGTACCTAGCAGATTTAGCGGTTCAGGAAGTGGGACTGATTTTACACTCACTATATCT
TCCCTGCAACCGGAGGATTTTGCAACATATTATTGTCAACAATATCTCTACCATCCCGCGACATTCGGGCAGGGCA
CAAAAGTAGAGATCAAACGAACAGTAGCAGCTCCGGAAATTGTGTTGACACAGTCTCCAGCCACCCTGTCTTTGTC
TCCAGGGGAAAGAGCCACCCTCTCCTGCAGGGCCAGTCAGAATATCAGCAACTTCTTAGCCTGGTACCAACACAAA
CCTGGCCAGGCTCCCAGGCTCCTCATCTATGATGCATCCATCAGGGCCACTGGCATCCCAGCCAGGTTCAGTGGCA
GTGGGTCTGGGACAGACTTCAGTCTCACCATCAGCAGCCTGGAGCCTGAAGATTTTGCAGTTTATTTCTGTCAGCA
GCGTTACAACTGGCTCACTTTCGGCGGAGGGACCAAGGTAGAGATCAAA
SEQ ID NO: 43-ATE-S-2H11 bispecific V_{L} domain amino acid sequence
DIQMTQSPSSLSASVGDRVTITCRASQDVSTAVAWYQQKPGKAPKLLIYSASFLYSGVPSRFSGSGSGTDFTLTISSLQPE
DFATYYCQQYLYHPATFGQGTKVEIKRTVAAPEIVLTQSPATLSLSPGERATLSCRASQNISNFLAWYQHKPGQAPRLLIY
DASIRATGIPARFSGSGSGTDFSLTISSLEPEDFAVYFCQQRYNWLTFGGGTKVEIK
SEQ ID NO: 44-V_{H} domain amino acid sequence of anti-CD-63 antibody
QVQLQESGPELVKPGASVKMSCKASGYTFTTYVIHWVKQKPGQGLEWIGYFDPNNDGTKYNERFKGKATLTSDRSSST
AYMELSSLTSEDSAVYYCARSRTYYDASMDYWGQGTSVTVSS
SEQ ID NO: 45-V_{L} domain amino acid sequence of anti-CD-63 antibody
DIWMTQSPSSLAVSPGEKVTMNCKSSQSVLYSSNQKNFLAWYQQKPGQSPKLLIYWASTRESGVPDRFTGSGGSGTD
FTLTISNIQTEDLAVYYCQQIFSSYTFGGGTKLELKR
SEQ ID NO: 46-V_{H} domain amino acid sequence of anti-HER2 antibody
EVQLVESGGGLVQPGGSLRLSCAASGENIKDTYIHWVRQAPGKGLEWVARIYPTNGYTRYADSVKGRFTISADTSKNTA
YLQMNSLRAEDTAVYYCSRWGGDGFYAMDYWGQGTLVTVSSASTKGPSVFPLA
SEQ ID NO: 47-V_{L} domain amino acid sequence of anti-HER2 antibody
DIQMTQSPSSLSASVGDRVTITCRASQDVNTAVAWYQQKPGKAPKLLIYSASFLYSGVPSRFSGSRSGTDFTLTISSLQPE
DFATYYCQQHYTTPPTFGQGTKVEIK
SEQ ID NO: 48-V_{H} domain amino acid sequence of anti-EpCAM antibody
EVQLVQSGPGLVQPGGSVRISCAASGYTFTNYGMNWVKQAPGKGLEWMGWINTYTGESTYADSFKGRFTFSLDTSA
SAAYLQINSLRAEDTAVYYCARFAIKGDYWGQGTLLTVSS
SEQ ID NO: 49-V_{L} domain amino acid sequence of anti-EpCAM antibody
DIQMTQSPSSLSASVGDRVTITCRSTKSLLHSNGITYLYWYQQKPGKAPKLLIYQMSNLASGVPSRFSSSGSGTDFTLTISS
LQPEDFATYYCAQNLEIPRTFGQGTKVELK
SEQ ID NO: 50-V_{H} domain amino acid sequence of anti-HER3 antibody
QVQLVQSGAEVKKPGASVKVSCKASGYTFTSHWMHWVRQAPGQGLEWIGEFNPSNGRTNYNEKFKSKATMTVDTS
TNTAYMELSSLRSEDTAVYYCASRDYDYDGRYFDYWGQGTLVTVSSASTKGPSVFPLA
SEQ ID NO: 51-V_{L} domain amino acid sequence of anti-HER3 antibody
DIQMTQSPSSLSASVGDRVTITCSASSSVTYMYWYQQKPGKAPKLLIYDTSNLASGVPSRFSGSGSGTDYTFTISSLQPED
IATYYCQQWSSHIFTFGQGTKVEIK
SEQ ID NO: 52-V_{H} domain amino acid sequence of anti-EGFR antibody
QVQLKQSGPGLVQPSQSLSITCTVSGFSLTNYGVHWVRQSPGKGLEWLGVIWSGGNTDYNTPFTSRLSINKDNSKSQV
FFKMNSLQSNDTAIYYCARALTYYDYEFAYWGQGTLVTVSAASTKGPSVFPL
SEQ ID NO: 53-V_{L} domain amino acid sequence of anti-EGFR antibody
DILLTQSPVILSVSPGERVSFSCRASQSIGTNIHWYQQRTNGSPRLLIKYASESISGIPSRFSGSGSGTDFTLSINSVESEDIA
DYYCQQNNNWPTTFGAGTKLELK

The claimed invention is:

1. A bispecific antibody comprising:
(A) a first antigen binding moiety specific for an exosomal protein, and
(B) a second antigen binding moiety specific for programmed cell death-ligand 1 (PD-L1).

2. The bispecific antibody of claim 1, wherein the second antigen binding moiety comprises:
(1) at least one of:
(a) a heavy chain complementarity-determining region (“CDR”)1 comprising the amino acid sequence of SEQ ID NO: 17;
(b) a heavy chain CDR2 comprising the amino acid sequence of SEQ ID NO: 18; and
(c) a heavy chain CDR3 comprising the amino acid sequence of SEQ ID NO: 19; and
(2) at least one of:
(a) a light chain CDR1 comprising the amino acid sequence of SEQ ID NO: 20;
(b) a light chain CDR2 comprising the amino acid sequence of SEQ ID NO: 21; and
(c) a light chain CDR3 comprising the amino acid sequence of SEQ ID NO: 22.

3. The bispecific antibody of claim 1, wherein the second antigen binding moiety comprises a V_{H} chain, or fragment thereof, and a V_{L} chain, or fragment thereof, of an anti PD-L1 antibody selected from atezolizumab, avelumab, durvalumab, and BMS-936559.

4. The bispecific antibody of any one of claims 1-3, wherein the first antigen binding moiety specifically binds an exosomal protein selected from EPN1, CD9, CD10, CD26, CD37, CD45/ICAM-1, CD63, CD69, CD81, EGFR, EGFRvIII, EpCAM, Flotillin-1, Glypican-1, HER2, HER3, HSP70, HSP90, and NKCC2.

5. The bispecific antibody of claim 4, wherein the first antigen binding moiety specifically binds an epitope on human EPN1.

6. The bispecific antibody of claim 5, wherein the first antigen binding moiety comprises:
(1) a variable heavy chain comprising the amino acid sequence of SEQ ID NO: 2, or a fragment thereof; and
(2) a variable light chain comprising the amino acid sequence of SEQ ID NO: 4, or a fragment thereof.

7. The bispecific antibody of claim 5, wherein the first antigen binding moiety comprises:
(1) a variable heavy chain comprising the amino acid sequence of SEQ ID NO: 6, or a fragment thereof; and
(2) a variable light chain comprising the amino acid sequence of SEQ ID NO: 8, or a fragment thereof.

8. The bispecific antibody of claim 5, wherein the first antigen binding moiety comprises:
(1) at least one of:
(a) a heavy chain CDR1 comprising the amino acid sequence of SEQ ID NO: 9;
(b) a heavy chain CDR2 comprising the amino acid sequence of SEQ ID NO: 10; and
(c) a heavy chain CDR3 comprising the amino acid sequence of SEQ ID NO:11; and
(2) at least one of:
(a) a light chain CDR1 comprising the amino acid sequence of SEQ ID NO: 12;
(b) a light chain CDR2 comprising the amino acid sequence of SEQ ID NO: 13, and
(c) a light chain CDR3 comprising the amino acid sequence of SEQ ID NO: 14.

9. The bispecific antibody of any one of claims 1-8, wherein the first and second first antigen binding moieties are connected directly or by a linker.

10. The bispecific antibody of claim 9, wherein the linker is selected from the group consisting of a chemical linker or a polypeptide linker.

11. The bispecific antibody of anyone of claims 1-10, wherein the bispecific antibody is selected from the group consisting of: a knob-into-hole derivative; SEED-IgG, DEKK mutated Fc, DVD-Ig, heteroFc-scFv, IgG-scFv, scFv2-Fc, scDB-Fc.

12. The bispecific antibody of any one of claims 1-10, wherein the bispecific antibody does not contain an Fc domain, and is selected from the group consisting of tandem scFv, diabody, Fab-scFv

13. The bispecific antibody of any one of claims 1-12, wherein the antibody is a fully human or humanized antibody.

14. A pharmaceutical composition comprising a bispecific antibody of any one of claims 1-13 and at least one pharmaceutically acceptable excipient.

15. A kit comprising at least one bispecific antibody of any one of claims 1-13 and at least one of: a suitable storage container, a pH buffered solution, and instructions for using the kit.

16. A method for treating cancer in a subject, comprising administering to the subject a therapeutically effective amount of a bispecific antibody of any one of claims 1-13.

17. The method of claim 16, wherein the bispecific antibody disrupts the suppression of anti-tumor activity by immune cells by targeting tumor-cell derived exosomes.

18. The method of claim 17, wherein the suppression of anti-tumor activity is mediated by CD8 suppressor T cells.

19. The method of any one of claims 16-18, wherein the subject is human.

20. The method of any one of claims 16-19, wherein about 0.5-20 mg/kg of the bispecific antibody is administered to the subject.


SEQUENCE LISTING (53 sequences)

SEQ ID NO: 1 - DNA, 354 bp, Homo sapiens
gactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcctccaccaagggcccatcggtcttccccctggcgc

SEQ ID NO: 2 - protein, 118 aa, Homo sapiens
LSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKKGPSVFPLA

SEQ ID NO: 3 - DNA, 299 bp, Homo sapiens
aagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaactgtggctgcaccatctgtcttcatct

SEQ ID NO: 4 - protein, 99 aa, Homo sapiens
RATLSCRASQNISNFLAWYQHKPGQAPRLLIYDASIRATGIPARFSGSGSGTDFSLTISSLEPEDFAVYFCQQRYNWLTFGGGTKVEIKRTVAAPSVFI

SEQ ID NO: 5 - DNA, 407 bp, Homo sapiens
acaggcgcgcactccgaggtgcagctggtggagtctgggggaggcttggtacagcctggggggtccctgagactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcctccaccaagggcccatc

SEQ ID NO: 6 - protein, 135 aa, Homo sapiens
EVQLVESGGGLVQPGGSLRLSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKGPSVFPL

SEQ ID NO: 7 - DNA, 346 bp, Homo sapiens
tcagatacctccggagaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaactgtggctg

SEQ ID NO: 8 - protein, 110 aa, Homo sapiens
EIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKRTVA

SEQ ID NO: 9 - protein, 6 aa, Homo sapiens
SIHSLN

SEQ ID NO: 10 - protein, 17 aa, Homo sapiens
YISSNSTTIYYADSVKG

SEQ ID NO: 11 - protein, 15 aa, Homo sapiens
DYYCTGGTCFFLPDL

SEQ ID NO: 12 - protein, 11 aa, Homo sapiens
RASQNISNFLA

SEQ ID NO: 13 - protein, 7 aa, Homo sapiens
DASIRAT

SEQ ID NO: 14 - protein, 8 aa, Homo sapiens
QQRYNWLT

SEQ ID NO: 15 - protein, 130 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLA

SEQ ID NO: 16 - protein, 111 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVA

SEQ ID NO: 17 - protein, 6 aa, Homo sapiens
SDSWIH

SEQ ID NO: 18 - protein, 14 aa, Homo sapiens
PYGGSTYYADSVKG

SEQ ID NO: 19 - protein, 11 aa, Homo sapiens
ARRHWPGGFDY

SEQ ID NO: 20 - protein, 11 aa, Homo sapiens
RASQDVSTAVA

SEQ ID NO: 21 - protein, 7 aa, Homo sapiens
SASFLYS

SEQ ID NO: 22 - protein, 9 aa, Homo sapiens
QQYLYHPAT

SEQ ID NO: 23 - protein, 132 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLA

SEQ ID NO: 24 - protein, 111 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLG

SEQ ID NO: 25 - protein, 133 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLA

SEQ ID NO: 26 - protein, 112 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVA

SEQ ID NO: 27 - protein, 135 aa, Homo sapiens
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLA

SEQ ID NO: 28 - protein, 110 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVA

SEQ ID NO: 29 - protein, 13 aa, Artificial Sequence
  Bispecific
ASTKGPSVFPLAP

SEQ ID NO: 30 - protein, 12 aa, Artificial Sequence
  Bispecific
TVAAPSVFIFPP

SEQ ID NO: 31 - protein, 5 aa, Artificial Sequence
  Bispecific
TVAAP

SEQ ID NO: 32 - DNA, 765 bp, Artificial Sequence
  Bispecific
gaggtgcagctggtggagtctgggggaggcttggtacagcctggggggtccctgagactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcgagcacaaaaggaccatctgtatttccactcgcccccgaagtacagctcgtagagtccggaggaggcctggtccaacctggtggttcccttcgactgtcatgtgccgcgtctggcttcactttttccgattcatggatacactgggtgaggcaagcacctggcaaaggtttggaatgggtggcctggatctcaccgtatgggggtagtacttattatgcggattcagtaaagggaagatttaccatttcagcggacacaagtaaaaataccgcctatttgcagatgaacagcctgcgagcggaagacactgctgtctattattgtgctagacgccactggcctggtggttttgactactgggggcagggcactttggtgaccgtttcctca

SEQ ID NO: 33 - protein, 255 aa, Artificial Sequence
  Bispecific
EVQLVESGGGLVQPGGSLRLSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKGPSVFPLAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 34 - DNA, 681 bp, Artificial Sequence
  Bispecific
gaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaacagtagcagctccgtcagtttttatttttcctccagatattcagatgacccagtccccgtcctctctctccgctagtgtaggtgatagagtgacaataacatgccgggccagccaggatgtatccacggcggtcgcgtggtaccagcagaaacctgggaaagcccccaaactgcttatttatagcgccagcttcttgtactcaggagtacctagcagatttagcggttcaggaagtgggactgattttacactcactatatcttccctgcaaccggaggattttgcaacatattattgtcaacaatatctctaccatcccgcgacattcgggcagggcacaaaagtagagatcaaacga

SEQ ID NO: 35 - protein, 227 aa, Artificial Sequence
  Bispecific
EIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKRTVAAPSVFIFPPDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 36 - DNA, 660 bp, Artificial Sequence
  Bispecific
gaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaacagtagcagctccggatattcagatgacccagtccccgtcctctctctccgctagtgtaggtgatagagtgacaataacatgccgggccagccaggatgtatccacggcggtcgcgtggtaccagcagaaacctgggaaagcccccaaactgcttatttatagcgccagcttcttgtactcaggagtacctagcagatttagcggttcaggaagtgggactgattttacactcactatatcttccctgcaaccggaggattttgcaacatattattgtcaacaatatctctaccatcccgcgacattcgggcagggcacaaaagtagagatcaaacga

SEQ ID NO: 37 - protein, 220 aa, Artificial Sequence
  Bispecific
EIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKRTVAAPDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 38 - DNA, 1243 bp, Artificial Sequence
  Bispecific
gaagtacagctcgtagagtccggaggaggcctggtccaacctggtggttcccttcgactgtcatgtgccgcgtctggcttcactttttccgattcatggatacactgggtgaggcaagcacctggcaaaggtttggaatgggtggcctggatctcaccgtatgggggtagtacttattatgcggattcagtaaagggaagatttaccatttcagcggacacaagtaaaaataccgcctatttgcagatgaacagcctgcgagcggaagacactgctgtctattattgtgctagacgccactggcctggtggttttgactactgggggcagggcactttggtgaccgtttcctcagccgcgagcacaaaaggaccatctgtatttccactcgcccccgaggtgcagctggtggagtctgggggaggcttggtacagcctggggggtccctgagaagtacagctcgtagagtccggaggaggcctggtccaacctggtggttcccttcgactgtcatgtgccgcgtctggcttcactttttccgattcatggatacactgggtgaggcaagcacctggcaaaggtttggaatgggtggcctggatctcaccgtatgggggtagtacttattatgcggattcagtaaagggaagatttaccatttcagcggacacaagtaaaaataccgcctatttgcagatgaacagcctgcgagcggaagacactgctgtctattattgtgctagacgccactggcctggtggttttgactactgggggcagggcactttggtgaccgtttcctcagccgcgagcacaaaaggaccatctgtatttccactcgcccccgaggtgcagctggtggagtctgggggaggcttggtacagcctggggggtccctgagactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcctccaccaagggcccatcggtc

SEQ ID NO: 39 - protein, 264 aa, Artificial Sequence
  Bispecific
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSAASTKGPSVFPLAPEVQLVESGGGLVQPGGSLRLSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKGPSV

SEQ ID NO: 40 - DNA, 681 bp, Artificial Sequence
  Bispecific
gatattcagatgacccagtccccgtcctctctctccgctagtgtaggtgatagagtgacaataacatgccgggccagccaggatgtatccacggcggtcgcgtggtaccagcagaaacctgggaaagcccccaaactgcttatttatagcgccagcttcttgtactcaggagtacctagcagatttagcggttcaggaagtgggactgattttacactcactatatcttccctgcaaccggaggattttgcaacatattattgtcaacaatatctctaccatcccgcgacattcgggcagggcacaaaagtagagatcaaacgaaccgtcgccgcaccatcagtttttatttttcctccagaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacga

SEQ ID NO: 41 - protein, 227 aa, Artificial Sequence
  Bispecific
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPEIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKR

SEQ ID NO: 42 - DNA, 657 bp, Artificial Sequence
  Bispecific
gatattcagatgacccagtccccgtcctctctctccgctagtgtaggtgatagagtgacaataacatgccgggccagccaggatgtatccacggcggtcgcgtggtaccagcagaaacctgggaaagcccccaaactgcttatttatagcgccagcttcttgtactcaggagtacctagcagatttagcggttcaggaagtgggactgattttacactcactatatcttccctgcaaccggaggattttgcaacatattattgtcaacaatatctctaccatcccgcgacattcgggcagggcacaaaagtagagatcaaacgaacagtagcagctccggaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaa

SEQ ID NO: 43 - protein, 219 aa, Artificial Sequence
  Bispecific
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPEIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIK

SEQ ID NO: 44 - protein, 120 aa, Homo sapiens
QVQLQESGPELVKPGASVKMSCKASGYTFTTYVIHWVKQKPGQGLEWIGYFDPNNDGTKYNERFKGKATLTSDRSSSTAYMELSSLTSEDSAVYYCARSRTYYDASMDYWGQGTSVTVSS

SEQ ID NO: 45 - protein, 114 aa, Homo sapiens
DIWMTQSPSSLAVSPGEKVTMNCKSSQSVLYSSNQKNFLAWYQQKPGQSPKLLIYWASTRESGVPDRFTGSGGSGTDFTLTISNIQTEDLAVYYCQQIFSSYTFGGGTKLELKR

SEQ ID NO: 46 - protein, 132 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLA

SEQ ID NO: 47 - protein, 107 aa, Homo sapiens [sequence omitted]

SEQ ID NO: 48 - protein, 116 aa, Homo sapiens
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 49 - protein, 112 aa, Homo sapiens
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 50 - protein, 133 aa, Homo sapiens
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLA

SEQ ID NO: 51 - protein, 106 aa, Homo sapiens [sequence omitted]

SEQ ID NO: 52 - protein, 130 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPL

SEQ ID NO: 53 - protein, 107 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK